(19) United States
(12) Patent Application Publication
(10) Pub. No.: US 2025/0346670 A1
(43) Pub. Date: Nov. 13, 2025

(54) METHODS OF TREATING CANCER BY ADMINISTERING A NEOADJUVANT PD-1 INHIBITOR

(71) Applicant: Regeneron Pharmaceuticals, Inc., Tarrytown, NY (US)

(72) Inventors: Elizabeth Miller, Tarrytown, NY (US); Israel Lowy, Dobbs Ferry, NY (US); Gavin Thurston, Briarcliff Manor, NY (US); Miriam Merad, New York, NY (US); Thomas Marron, New York, NY (US); Myron Schwartz, New York, NY (US)

(21) Appl. No.: 18/264,799

(22) PCT Filed: Feb. 10, 2022
(86) PCT No.: PCT/US2022/015950

§ 371 Date: Aug. 9, 2023

Related U.S. Application Data
(60) Provisional application No. 63/148,239, filed on Feb. 11, 2021.
(60) Provisional application No. 63/166,183, filed on Mar. 25, 2021.
(60) Provisional application No. 63/222,727, filed on Jul. 16, 2021.

(51) Int. Cl. C07K 16/28; A61K 39/00; A61K 39/395; A61K 45/06; A61P 35/00

(52) U.S. Cl. CPC C07K 16/2818; A61K 39/3955; A61K 45/06; A61P 35/00; A61K 2039/54; A61K 2039/545; A61K 2039/55

(57) ABSTRACT

The present disclosure provides methods for treating, reducing the severity of, inhibiting the growth of a tumor, or inducing necrosis of a tumor, wherein the method includes selecting a patient with cancer (e.g., liver cancer, lung cancer, or head and neck cancer) in need thereof and administering to the patient a therapeutically effective amount of a programmed death 1 (PD-1) inhibitor (e.g., cemiplimab or abioequivalent thereof) as neoadjuvant therapy followed by surgical resection and optional administration of a programmed death 1 (PD-1) inhibitor (e.g., cemiplimab or a bioequivalent thereof) as post-surgery adjuvant therapy. In certain embodiments, the liver cancer is hepatocellular carcinoma (HCC), the lung cancer is non-small cell lung cancer (NSCLC), or the head and neck cancer is head and neck squamous cell carcinoma (HNSCC).

### CROSS-REFERENCE TO RELATED APPLICATIONS

This application is a U.S. national phase application under 35 U.S.C. § 371 of International Patent Application No. PCT/US2022/015950 filed Feb. 10, 2022, which claims the benefit of priority to U.S. Provisional Patent Application No. 63/222,727 filed Jul. 16, 2021, U.S. Provisional Patent Application No. 63/166,183 filed Mar. 25, 2021, and U.S. Provisional Patent Application No. 63/148,239 filed Feb. 11, 2021, the disclosures of all of which are hereby incorporated by reference in their entireties.

### SEQUENCE LISTING

The sequence listing of the present application is submitted electronically as a computer readable sequence listing in ASCII format with a file name of “SeqList10911.txt,” a creation date of Dec. 21, 2023, and a size of 8,605 bytes. This sequence listing submitted is part of the specification and is hereby incorporated by reference in its entirety.

### FIELD

The present disclosure relates to methods of treating or inhibiting the growth of a tumor, including selecting a patient with cancer in need thereof and administering to the patient a therapeutically effective amount of a programmed death 1 (PD-1) inhibitor (e.g., cemiplimab or a bioequivalent thereof) as neoadjuvant therapy followed by surgical resection.

### BACKGROUND

Non-small cell lung cancer (NSCLC), liver cancer, and head and neck squamous cell carcinoma (HNSCC) are some of the leading causes of cancer deaths worldwide.

NSCLC causes the most cancer deaths in men and women, and the majority of patients do not achieve significant clinical benefit from the combination of PD-1/PD-L1 blockade and chemotherapy. Until recently, roughly three-quarters of lung cancer was diagnosed as Stage 4 disease. Computed tomography (CT) screening has increased the number of earlier stage, potentially curable tumors detected. Yet, despite increasingly identifying NSCLC at earlier stages, operable Stage 1-3 lung cancer—which recurs in the majority of patients—has seen few, significant improvements in treatment approaches. Given high rates of recurrence and the paucity of effective treatments, better approaches are needed.

HNSCC is the sixth most common malignancy worldwide, and the rates of HNSCC have increased steadily since the 1980s, in part due to the rise in human papilloma virus (HPV) infection in the oropharynx, so that currently, roughly half of the cases of HNSCC that occur in the developed world are attributable to HPV infection, while the latter only accounts for 10-20% of cases in the developing world (Torre et al., A Cancer Journal for Clinicians, 2015; 65(2):87-108; Chaturvedi et al., Journal of Clinical Oncology: Official Journal of the American Society of Clinical Oncology, 2008; 26(4):612-619; de Martel et al., The Lancet Oncology, 2012; 13(6):607-615). Patients with HPV-related disease, across stages, have improved survival outcomes when compared to HPV-negative tumors, which are commonly associated with tobacco and alcohol exposure (Ang, 2010). Early-stage disease is typically treated with surgery and/or radiation, and these patients have a good prognosis, with 5-year overall survival (OS) of 70-90%, but patients with locoregionally advanced disease who often receive chemotherapy in addition to locoregional therapies (surgery and/or radiation) have a dismal 30% 5-year OS, worse still in those with HPV-negative disease (Blanchard et al., Radiother Oncol, 2011; 100(1):33-40). Hence, improved treatments for NSCLC are needed.

Liver cancer, particularly hepatocellular carcinoma (HCC), accounts for the second most cancer deaths in men worldwide (Ferlay et al., Int J Cancer, 2015; 136(5):E359-E386), and is the most rapidly rising cause of cancer mortality in the United States, with more than 30,000 new cases per year (Siegel et al., A Cancer Journal for Clinicians, 2013; 63(1):11-30; Torre et al., A Cancer Journal for Clinicians, 2015; 65(2):87-108). HCC accounts for 75%-85% of primary liver cancer cases, which was the third leading cause of cancer death worldwide in 2020. (Sung et al., CA Cancer J Clin, 2021). The recommended first-line treatment for very early/early-stage HCC is surgery, which includes hepatic resection and transplantation, or radiofrequency ablation (RFA) in patients with preserved liver function, and results have improved with advances in surgical techniques and perioperative care. However, there is a high incidence of postoperative recurrence and cancer-related deaths. (European Association for the Study of the Liver. J Hepatol. 2018; 69:182-236; Poon et al., Ann Surg. 2000; 232:10-24; Chan et al., Liver Transpl. 2013; 19:411-419). Negative margins are usually observed at the time of surgical resection; however, it is believed that HCC recurs as a result of residual micrometastases that persist after resection, highlighting the potential benefit of neoadjuvant therapy in improving HCC outcomes. There is no standard recommended treatment in the neoadjuvant setting (European Association for the Study of the Liver. J Hepatol. 2018; 69:182-236; Akateh C et al. World J Gastroenterol 2019; 25:3704-3721). And no neoadjuvant or adjuvant therapies have demonstrated a reduction in risk of recurrence or a proven survival benefits in patients with HCC. While immunotherapy combinations have changed the prognosis of patients with advanced HCC, the majority of patients still perish from this disease.

HCC typically presents in advanced stages, at which time surgery is not an option. Hence, the usual prognosis for HCC is poor because only 10-20% of hepatocellular carcinomas can be removed completely by surgery. If the cancer cannot be completely removed, the disease is often fatal within three to six months. Additionally, PD-1 and PD-L1 are generally overexpressed in HCC, and high PD-L1 expression by tumor cells has been associated with significantly poorer prognosis.

The treatment of choice for patients with HCC and preserved liver function is surgical resection, with hepatic resection being the accepted treatment of early-stage HCC. However, postsurgical tumor intrahepatic recurrence is common, with early (within 2 years) recurrence being observed in approximately 50% of cases (Franssen et al., Ann Surg, 2014; 260(4):650-656; Tabrizian et al., Ann Surg., 2015; 261(5):947-955). In fact, the majority of tumors recur despite surgery and no perioperative intervention has demonstrated a survival advantage. Since negative margins are usually observed at the time of surgical resection, it is believed that HCC recurrence occurs as a result of micrometastases that persist after resection. Chemotherapy usually has no role in the management of HCC. Targeted agents such as sorafenib have shown some survival benefit in patients with unresectable disease, but a large international trial of adjuvant sorafenib treatment showed no benefit (Bruix et al., The Lancet Oncology, 2015; 16(13):1344-1354). Thus, there remains a significant need for a safe and effective therapy for treating liver cancer, including HCC.

### SUMMARY

In one aspect, the disclosed technology relates to a method of treating or inhibiting the growth of a tumor, comprising: (a) selecting a patient with liver cancer; (b) administering to the patient a therapeutically effective amount of a neoadjuvant programmed death-1 (PD-1) inhibitor, wherein the neoadjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof; and (c) after step (b), surgically resecting the liver cancer tumor. In some embodiments, the liver cancer is resectable. In some embodiments, the liver cancer is selected from hepatocellular carcinoma (HCC), fibrolamellar carcinoma, cholangiocarcinoma, angiosarcoma, and hepatoblastoma. In some embodiments, the liver cancer is HCC. In some embodiments, the liver cancer is recurrent. In some embodiments, the liver cancer is metastatic. In some embodiments, the patient has liver cancer for which the intent of surgery would be curative. In some embodiments, the patient has a chronic viral infection that has been treated and controlled with an anti-viral therapy and wherein the chronic viral infection comprises HIV, HBV, HCV, or a combination thereof. In some embodiments, the patient has squamous or non-squamous liver cancer. In some embodiments, the patient has PD-L1 expression in: 1% of liver cancer cells. In some embodiments, surgical resection is performed more than 28 days after step (b).

In some embodiments, the administered neoadjuvant anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8. In some embodiments, the administered neoadjuvant anti-PD-1 antibody comprises a HCVR comprising an amino acid sequence of SEQ ID NO: 1. In some embodiments, the administered neoadjuvant anti-PD-1 antibody comprises a LCVR comprising an amino acid sequence of SEQ ID NO: 2. In some embodiments, the administered neoadjuvant anti-PD-1 antibody comprises a HCVR/LCVR amino acid sequence pair of SEQ ID NOs: 1/2. In some embodiments, the administered neoadjuvant anti-PD-1 antibody comprises a heavy chain and a light chain, wherein the heavy chain has an amino acid sequence of SEQ ID NO: 9. In some embodiments, the administered neoadjuvant anti-PD-1 antibody comprises a heavy chain and a light chain, wherein the light chain has an amino acid sequence of SEQ ID NO: 10. In some embodiments, the administered neoadjuvant anti-PD-1 antibody comprises a heavy chain and a light chain, wherein the heavy chain has an amino acid sequence of SEQ ID NO: 9 and the light chain has an amino acid sequence of SEQ ID NO: 10. In some embodiments, the administered neoadjuvant anti-PD-1 antibody is cemiplimab.

In some embodiments, the administered neoadjuvant PD-1 inhibitor is an anti-PD-1 antibody comprising a HCVR with 90%, 95%, 97%, or 98% sequence identity to SEQ ID NO: 1. In some embodiments, the administered neoadjuvant PD-1 inhibitor is an anti-PD-1 antibody comprising a LCVR with 90%, 95%, 97%, or 98% sequence identity to SEQ ID NO: 2. In some embodiments, the administered neoadjuvant PD-1 inhibitor is an anti-PD-1 antibody comprising a HCVR with 90%, 95%, 97%, or 98% sequence identity to SEQ ID NO: 1, and a LCVR with 90%, 95%, 97%, or 98% sequence identity to SEQ ID NO: 2.

In another aspect, the disclosed methods further include: (d) after step (c), administering to the patient a therapeutically effective amount of an adjuvant programmed death-1 (PD-1) inhibitor, wherein the adjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof. In some embodiments, the administered adjuvant anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

In some embodiments, the administered adjuvant anti-PD-1 antibody comprises a HCVR comprising an amino acid sequence of SEQ ID NO: 1. In some embodiments, the administered adjuvant anti-PD-1 antibody comprises a LCVR comprising an amino acid sequence of SEQ ID NO: 2. In some embodiments, the administered adjuvant anti-PD-1 antibody comprises a HCVR/LCVR amino acid sequence pair of SEQ ID NOs: 1/2.

In some embodiments, the administered adjuvant anti-PD-1 antibody comprises a heavy chain and a light chain, wherein the heavy chain has an amino acid sequence of SEQ ID NO: 9. In some embodiments, the administered adjuvant anti-PD-1 antibody comprises a heavy chain and a light chain, wherein the light chain has an amino acid sequence of SEQ ID NO: 10. In some embodiments, the administered adjuvant anti-PD-1 antibody comprises a heavy chain and a light chain, wherein the heavy chain has an amino acid sequence of SEQ ID NO: 9 and the light chain has an amino acid sequence of SEQ ID NO: 10.

In some embodiments, the administered adjuvant PD-1 inhibitor is an anti-PD-1 antibody comprising a HCVR with 90%, 95%, 97%, or 98% sequence identity to SEQ ID NO: 1. In some embodiments, the administered adjuvant PD-1 inhibitor is an anti-PD-1 antibody comprising a LCVR with 90%, 95%, 97%, or 98% sequence identity to SEQ ID NO: 2. In some embodiments, the administered adjuvant PD-1 inhibitor is an anti-PD-1 antibody comprising a HCVR with 90%, 95%, 97%, or 98% sequence identity to SEQ ID NO: 1, and a LCVR with 90%, 95%, 97%, or 98% sequence identity to SEQ ID NO: 2.

In another aspect, the disclosed methods lead to necrosis of the resected tumor, promote tumor regression, reduce tumor cell load, reduce tumor burden, and/or prevent tumor recurrence in the patient. In some embodiments, the method leads to more than 50% necrosis of the resected tumor. In some embodiments, the method leads to more than 70% necrosis of the resected tumor.

In another aspect, the disclosed methods further include administering to the patient an additional therapeutic agent or therapy selected from one or more of: an anti-viral therapy, photodynamic therapy, a programmed death ligand 1 (PD-L1) inhibitor, a lymphocyte activation gene 3 (LAG3) inhibitor, a cytotoxic T-lymphocyte-associated protein 4 (CTLA-4) inhibitor, a glucocorticoid-induced tumor necrosis factor receptor (GITR) agonist, a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand, a CD20 inhibitor, an indoleamine-2,3-dioxygenase (IDO) inhibitor, a CD28 activator, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an agonist to a co-stimulatory receptor, an antibody to a tumor-specific antigen, a vaccine, an adjuvant to increase antigen presentation, an oncolytic virus, a cytotoxin, a chemotherapeutic agent, platinum-based chemotherapy, a tyrosine kinase inhibitor, an IL-6R inhibitor, an IL-4R inhibitor, an IL-10 inhibitor, a cytokine, an antibody drug conjugate (ADC), chimeric antigen receptor T cells, an anti-inflammatory drug, and a dietary supplement.

In some embodiments, the neoadjuvant PD-1 inhibitor is administered as one or more doses, wherein each dose is administered every two weeks, three weeks, four weeks, five weeks or six weeks. In some embodiments, the neoadjuvant PD-1 inhibitor is administered as two or more doses, wherein each dose is administered every three weeks. In some embodiments, the neoadjuvant PD-1 inhibitor is administered at a dose of 5 mg to 1000 mg. In some embodiments, the neoadjuvant PD-1 inhibitor is administered at a dose of 200 mg, 250 mg, 350 mg, 400 mg, 500 mg, 600 mg, 750 mg, 800 mg, or 1000 mg. In some embodiments, the neoadjuvant PD-1 inhibitor is administered at a dose of 1 mg/kg to 20 mg/kg of the patient's body weight. In some embodiments, the neoadjuvant PD-1 inhibitor is administered at a dose of 1 mg/kg, 3 mg/kg or 10 mg/kg of the patient's body weight. In some embodiments, the neoadjuvant PD-1 inhibitor is administered intravenously, or subcutaneously.

In some embodiments, the adjuvant PD-1 inhibitor is administered as one or more doses, wherein each dose is administered every two weeks, three weeks, four weeks, five weeks or six weeks. In some embodiments, each dose of the adjuvant PD-1 inhibitor is administered every three weeks. In some embodiments, the adjuvant PD-1 inhibitor is administered at a dose of 5 mg to 1000 mg. In some embodiments, the adjuvant PD-1 inhibitor is administered at a dose of 200 mg, 250 mg, 350 mg, 400 mg, 500 mg, 600 mg, 750 mg, 800 mg, or 1000 mg. In some embodiments, the adjuvant PD-1 inhibitor is administered at a dose of 1 mg/kg to 20 mg/kg of the patient's body weight. In some embodiments, the adjuvant PD-1 inhibitor is administered at a dose of 1 mg/kg, 3 mg/kg or 10 mg/kg of the patient's body weight. In some embodiments, the adjuvant PD-1 inhibitor is administered intravenously, or subcutaneously.

In another aspect, the disclosed technology relates to a programmed death 1 (PD-1) inhibitor for use in a method of treating or inhibiting the growth of a tumor, the method comprising: (a) selecting a patient with liver cancer; (b) administering to the patient a therapeutically effective amount of a neoadjuvant programmed death-1 (PD-1) inhibitor, wherein the neoadjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof; and (c) after step (b), surgically resecting the liver cancer tumor.

In another aspect, the disclosed technology relates to a kit including a programmed death 1 (PD-1) inhibitor in combination with written instructions for use of a therapeutically effective amount of the PD-1 inhibitor for treating or inhibiting the growth of a tumor in a patient with liver cancer.

In another aspect, the disclosed technology relates to a method of treating or inhibiting the growth of a tumor, including: (a) selecting a patient with lung cancer; (b) administering to the patient a therapeutically effective amount of a neoadjuvant programmed death-1 (PD-1) inhibitor, wherein the neoadjuvant PD-1 inhibitor is an antibody that binds specifically to PD-1 and includes three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof; and (c) after step (b), surgically resecting the lung cancer tumor. In some embodiments, the lung cancer is non-small cell lung cancer. In some embodiments, the administered neoadjuvant anti-PD-1 antibody includes HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8. In some embodiments, the administered neoadjuvant anti-PD-1 antibody includes a HCVR/LCVR amino acid sequence pair of SEQ ID NOs: 1/2. In some embodiments, the method further includes: (d) after step (c), administering to the patient a therapeutically effective amount of an adjuvant programmed death-1 (PD-1) inhibitor, wherein the adjuvant PD-1 inhibitor is an antibody that binds specifically to PD-1 and includes three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof.

In another aspect, the disclosed technology includes a method of treating or inhibiting the growth of a tumor, including: (a) selecting a patient with head and neck cancer; (b) administering to the patient a therapeutically effective amount of a neoadjuvant programmed death-1 (PD-1) inhibitor, wherein the neoadjuvant PD-1 inhibitor is an antibody that binds specifically to PD-1 and includes three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof; and (c) after step (b), surgically resecting the head and neck cancer tumor. In some embodiments, the head and neck cancer is head and neck squamous cell carcinoma. In some embodiments, the administered neoadjuvant anti-PD-1 antibody includes HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8. In some embodiments, the administered neoadjuvant anti-PD-1 antibody includes a HCVR/LCVR amino acid sequence pair of SEQ ID NOs: 1/2. In some embodiments, the method further includes: (d) after step (c), administering to the patient a therapeutically effective amount of an adjuvant programmed death-1 (PD-1) inhibitor, wherein the adjuvant PD-1 inhibitor is an antibody that binds specifically to PD-1 and includes three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof.

### BRIEF DESCRIPTION OF THE FIGURES

FIG. 1 is a graph showing tumor necrosis and tumor size change from baseline in patients enrolled in the study described in Example 2.

FIG. 2 shows representative MRI images in a responder and a non-responder with corresponding hematoxylin and eosin images in relation to the study described in Example 2.

FIG. 3 is a graph showing pathology necrosis change by tumor-infiltrating lymphocyte (TIL) change in relation to the study described in Example 2.

FIG. 4 shows tissue analysis and multiplex immunohistochemistry (IHC) in relation to the study described in Example 2.

FIG. 5A is a graph showing response (tumor size change from baseline) as assessed by standard imaging, and tumor necrosis as assessed by pathologic examination and imaging for patients enrolled in the study described in Example 3. *Denotes a patient for whom MRI was contraindicated, so MRI-based analysis of necrosis not possible; pathologic necrosis was 0%.

FIG. 5B is a graph showing that estimated necrosis defined by MRI was strongly correlated with pathologic assessment of necrosis at surgery for patients enrolled in the study described in Example 3.

FIG. 6 provides graphs showing comparative response assessment of surgical pathology, RECIST, and necrosis on imaging of patients enrolled in the study described in Example 3.

FIG. 7A shows, in the left panel, results of manual scoring of TLS-like abundance by pathologists; TLS-like are characterized as high-density aggregates of lymphocytes within tumor lesions; and pathologist interpretation revealed that these aggregates were identified in the tumor tissue of each responder; and, in the right panel, TIL infiltration within tumor lesions with <50% necrosis or ≥50% necrosis, based on the H&E analysis of pathologists; 100% of the patients with ≥50% necrosis presented the highest score of TIL infiltration versus 21% of the patients with low tumor necrosis; and 29% of these patients did not have TIL infiltration, in relation to the study described in Example 3.

FIG. 7B is a histogram representing the percentage of CD8+ T cells among CD45+ cells in tumor lesion and adjacent tissue of eight patients (four with <50% tumor necrosis and four with ≥50% tumor necrosis). Cells were analysed by mass cytometry (CyTOF). CD8+ T cells are significantly enriched in the tumor of patients with high levels of necrosis versus the patients with low tumor necrosis, whereas the adjacent tissue is not differentially populated, in relation to the study described in Example 3.

FIG. 7C is a graph showing mean density of each immune subset at baseline and at resection in patients with ≥50% necrosis, in relation to the study described in Example 3. Sections from baseline or resection tumor FFPE samples were immuno-stained as shown in (D). Immune subsets are defined as T cells (CD3+, CD8+ T cells (CD3+, CD8+), CD4conv (CD3+, CD8−, FOXP3−), Tregs (CD3+, FOXP3+), myeloid cells (CD68+), and B cells (CD20+). The error bars indicate one standard error of the mean.

FIG. 7D shows Bulk RNA sequencing (BulkSeq) of biopsy cores and tumor resection of 11 patients (7 patients with little to no necrosis on resection [all <50% necrosis] and 4 patients with ≥50% necrosis, in relation to the study described in Example 3. Publicly available gene signatures associated with CD8+ T cells and Tregs, as well as exhaustion, cytotoxic, and naive programs were quantified in bulk patient samples. Statistical significance was defined by Wilcox signed-rank test. p-values were Bonferroni corrected to address multiple hypothesis testing.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

### Methods of Treating or Inhibiting Growth of Cancer

The present disclosure includes methods for treating or inhibiting the growth of a tumor comprising selecting a patient with liver cancer, lung cancer, or head and neck cancer and administering to the patient in need thereof a PD-1 inhibitor, such as cemiplimab or a bioequivalent thereof, wherein the PD-1 inhibitor is administered as neoadjuvant therapy prior to treating the patient with surgery (e.g., hepatic resection). In certain embodiments, the disclosed methods further include administering to the subject a PD-1 inhibitor (e.g., cemplimab or a bioequivalent thereof) as an adjuvant therapy after completion of surgery for treating liver cancer, lung cancer, or head and neck cancer. In certain embodiments, the disclosed methods include administering to a subject in need thereof a PD-1 inhibitor (e.g., cemplimab or a bioequivalent thereof) as a neoadjuvant treatment prior to planned surgery for treating liver cancer, lung cancer, or head and neck cancer, and subsequently administering to the patient a PD-1 inhibitor as adjuvant therapy post-surgery.

As used herein, “liver cancer” refers to cancer of the liver, such as hepatocellular carcinoma, fibrolamellar carcinoma, cholangiocarcinoma, angiosarcoma, and hepatoblastoma. In some embodiments, the liver cancer is hepatocellular carcinoma (HCC). In some embodiments, the liver cancer is resectable and recurrent. In some embodiments, the liver cancer is metastatic. In some embodiments, the patient is a surgical candidate for resection of the liver cancer tumor. In some embodiments, the patient has liver cancer for which the intent of surgery would be curative.

As used herein, “lung cancer” refers to cancer of the lung, such as non-small cell lung cancer (NSCLC) (e.g., advanced NSCLC, stage IIIB, stage IIIC, or stage IV squamous or non-squamous NSCLC, adenocarcinoma, squamous cell carcinoma, or large cell carcinoma), adenosquamous carcinoma, and sarcomatoid carcinoma. In some embodiments, the lung cancer is non-small cell lung cancer. In some embodiments, the lung cancer is squamous non-small cell lung cancer. In some embodiments, the lung cancer is non-squamous non-small cell lung cancer. In some embodiments, the lung cancer is locally advanced, recurrent or metastatic lung cancer.

As used herein, the term “head and neck cancer” refers to cancer of the mouth, sinuses, nose or throat—e.g., head and neck squamous cell carcinoma (HNSCC).

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to increase duration of survival of the subject. In many embodiments, the terms “tumor”, “lesion,” “tumor lesion,” “cancer,” and “malignancy” are used interchangeably and refer to one or more cancerous growths.

As used herein, the term “recurrent” refers to a frequent or repeated diagnosis of liver cancer, lung cancer, or head and neck cancer in a patient or a frequent or repeated occurrence of individual tumors, such as primary tumors and/or new tumors that may represent recurrence of a prior tumor. In certain embodiments, administration of the PD-1 inhibitor inhibits the recurrence of a liver cancer, lung cancer, or head and neck cancer tumor in the patient.

As used herein, the expression “a subject in need thereof” means a human or non-human mammal that exhibits one or more symptoms or indications of liver cancer, lung cancer, or head and neck cancer), and/or who has been diagnosed with liver cancer, lung cancer, or head and neck cancer, and who needs treatment for the same. In many embodiments, the terms “subject” and “patient” are used interchangeably. The expression includes subjects with primary, established, or recurrent tumors (advanced malignancies). In specific embodiments, the expression includes human subjects that have and/or need treatment for recurrent but not metastatic liver cancer, lung cancer, or head and neck cancer. In certain embodiments, the expression includes patients with a solid tumor that is resistant to or refractory to or is inadequately controlled by prior therapy (e.g., surgery or treatment with an anti-cancer agent other than cemiplimab or a bioequivalent thereof). In certain embodiments, the expression includes subjects with liver cancer, lung cancer, or head and neck cancer who are candidates for curative surgery.

In certain embodiments, the methods of the present disclosure are used for treating a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and blastomas.

In certain embodiments, the disclosed methods include administering a therapeutically effective amount of a PD-1 inhibitor (e.g., cemplimab or a bioequivalent thereof) in combination with an additional therapeutic agent or therapy. The additional therapeutic agent or therapy may be administered for increasing anti-tumor efficacy, for reducing toxic effects of one or more therapies and/or for reducing the dosage of one or more therapies. In various embodiments, the additional therapeutic agent or therapy may include one or more of: an anti-viral therapy (e.g., cidofovir), photodynamic therapy, a programmed death ligand 1 (PD-L1) inhibitor (e.g., an anti-PD-L1 antibody as disclosed in US 2015/0203580 or atezolizumab), a lymphocyte activation gene 3 (LAG3) inhibitor (e.g., an anti-LAG3 antibody), a cytotoxic T-lymphocyte-associated protein 4 (CTLA-4) inhibitor (e.g., ipilimumab), a glucocorticoid-induced tumor necrosis factor receptor (GITR) agonist (e.g., an anti-GITR antibody), a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand (e.g., an antibody to CD-28, 2B4, LY108, LAIR1, ICOS, CD160 or VISTA), a CD20 inhibitor (e.g., an anti-CD20 antibody, or a bispecific CD3/CD20 antibody), an indoleamine-2,3-dioxygenase (IDO) inhibitor, a CD28 activator, a vascular endothelial growth factor (VEGF) antagonist (e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, pazopanib, or ramucirumab)), an angiopoietin-2 (Ang2) inhibitor, a transforming growth factor beta (TGFβ) inhibitor, an epidermal growth factor receptor (EGFR) inhibitor (e.g., erlotinib, cetuximab), an agonist to a co-stimulatory receptor (e.g., an agonist to CD28, 4-1BB, or OX40), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin or a cancer vaccine), an adjuvant to increase antigen presentation (e.g., granulocyte-macrophage colony-stimulating factor), an oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), platinum-based chemotherapy (e.g., platinum-doublet chemotherapy), a tyrosine kinase inhibitor (e.g., lenvatinib, regorafenib, and cabozantinib), an IL-6R inhibitor, an IL-4R inhibitor, an IL-10 inhibitor, a cytokine such as IL-2, IL-7, IL-12, IL-21, and IL-15, an antibody drug conjugate (ADC) (e.g., anti-CD19-DM4 ADC, and anti-DS6-DM4 ADC), chimeric antigen receptor T cells (e.g., CD19-targeted T cells), an anti-inflammatory drug such as a corticosteroid, a non-steroidal anti-inflammatory drug (NSAID), and a dietary supplement such as an antioxidant.

As used herein, the term “anti-viral therapy” refers to any agent, drug or therapy used to treat, prevent, or ameliorate a viral infection in a host subject, including but not limited to: zidovudine, lamivudine, abacavir, ribavirin, lopinavir, efavirenz, cobicistat, tenofovir, rilpivirine, analgesics, corticosteroids, and combinations thereof. In the context of the present disclosure, chronic viral infections include those caused by viruses, including but not limited to: human immunodeficiency virus (HIV), hepatitis B virus (HBV), and hepatitis C virus (HCV).

In certain embodiments, administering to a subject with liver cancer a therapeutically effective amount of a PD-1 inhibitor (e.g., cemplimab or a bioequivalent thereof) leads to increased inhibition of tumor growth—e.g., greater tumor regression in the treated subject. In certain embodiments, administering to a subject with liver cancer a therapeutically effective amount of a PD-1 inhibitor (e.g., cemplimab or a bioequivalent thereof) leads to necrosis of the resected tumor, e.g., more than 50% necrosis, more than 60% necrosis, more than 70% necrosis, or more than 80% necrosis. In certain embodiments, administering to a subject with liver cancer a therapeutically effective amount of a PD-1 inhibitor (e.g., cemplimab or a bioequivalent thereof) leads to increased tumor regression, tumor shrinkage and/or disappearance.

In certain embodiments, the administration of a PD-1 inhibitor leads to one or more of: (i) delay to surgery, e.g., surgery more than 28 days after the end of the cycle of the last dose of neoadjuvant PD-1 inhibitor (e.g., cemplimab or a bioequivalent thereof); (ii) delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years in the treated subject, as compared to an untreated subject or a subject treated with surgical resection alone; (iii) increased disease-free survival (DFS) from date of surgery until recurrence of tumor or death, as compared to an untreated subject or a subject treated with surgical resection alone; and (iv) improved overall response rate, complete response, or partial response, as compared to an untreated subject or a subject treated with surgical resection alone. In certain embodiments, administering to a subject with liver cancer a therapeutically effective amount of a PD-1 inhibitor (e.g., cemplimab or a bioequivalent thereof) prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months as compared to an untreated subject or a subject treated with surgical resection alone. In certain embodiments, administering to a subject with lung cancer or head and neck cancer a therapeutically effective amount of a PD-1 inhibitor (e.g., cemplimab or a bioequivalent thereof) prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months as compared to an untreated subject or a subject treated with surgical resection alone.

In certain embodiments, administering to a subject with liver cancer a therapeutically effective amount of a PD-1 inhibitor (e.g., cemplimab or a bioequivalent thereof) leads to increased overall survival (OS) or progression-free survival (PFS) of the subject as compared to a subject treated with surgical resection alone. In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with surgical resection alone. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with surgical resection alone.

In certain embodiments, administering to a subject with lung cancer or head and neck cancer a therapeutically effective amount of a PD-1 inhibitor (e.g., cemplimab or a bioequivalent thereof) leads to increased overall survival (OS) or progression-free survival (PFS) of the subject as compared to a subject treated with surgical resection alone. In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with surgical resection alone. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with surgical resection alone.

### PD-1 Inhibitors

The methods disclosed herein include administering a therapeutically effective amount of a PD-1 inhibitor, wherein the PD-1 inhibitor is cemiplimab (also known as REGN2810; LIBTAYO®) or a bioequivalent thereof. As used herein, the term “bioequivalent” refers to anti-PD-1 antibodies or PD-1-binding proteins or fragments thereof that are pharmaceutical equivalents or pharmaceutical alternatives whose rate and/or extent of absorption do not show a significant difference with that of cemiplimab when administered at the same molar dose under similar experimental conditions, either single dose or multiple dose. In the context of the present disclosure, the term “bioequivalent” includes antigen-binding proteins that bind to PD-1 and do not have clinically meaningful differences with cemiplimab with respect to safety, purity and/or potency.

The term “antibody,” as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}—C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the PD-1 inhibitor is an anti-PD-1 antibody (e.g., cemiplimab) comprising three heavy chain complementarity determining regions (HCDRs) of a heavy chain variable region (HCVR) comprising the amino acid sequence of SEQ ID NO: 1 and three light chain complementarity determining regions (LCDRs) of a light chain variable region (LCVR) comprising the amino acid sequence of SEQ ID NO: 2. According to certain embodiments, the anti-PD-1 antibody (e.g., cemiplimab) comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In certain embodiments, the anti-PD-1 antibody (e.g., cemiplimab) comprises an HCVR comprising SEQ ID NO: 1 and an LCVR comprising SEQ ID NO: 2. In certain embodiments, the anti-PD-1 antibody (e.g., cemiplimab) comprises a heavy chain comprising the amino acid sequence of SEQ ID NO: 9 and a light chain comprising the amino acid sequence of SEQ ID NO: 10.

According to certain embodiments, a bioequivalent of cemiplimab is an anti-PD-1 antibody comprising a HCVR having 90%, 95%, 97% or 98% sequence identity to SEQ ID NO: 1. According to certain embodiments, a bioequivalent of cemiplimab is an anti-PD-1 antibody comprising a LCVR having 90%, 95%, 97% or 98% sequence identity to SEQ ID NO: 2. According to certain embodiments, a bioequivalent of cemiplimab is an anti-PD-1 antibody comprising a HCVR having 90%, 95%, 97% or 98% sequence identity to SEQ ID NO: 1, and a LCVR having 90%, 95%, 97% or 98% sequence identity to SEQ ID NO: 2. Sequence identity may be measured by methods known in the art (e.g., GAP, BESTFIT, and BLAST).

According to certain embodiments, a bioequivalent of cemiplimab is an anti-PD-1 antibody comprising a HCVR comprising an amino acid sequence of SEQ ID NO: 1 having no more than 5 amino acid substitutions. According to certain embodiments, a bioequivalent of cemiplimab is an anti-PD-1 antibody comprising a LCVR comprising an amino acid sequence of SEQ ID NO: 2 having no more than 2 amino acid substitutions. According to certain embodiments, a bioequivalent of cemiplimab is an anti-PD-1 antibody comprising a HCVR comprising an amino acid sequence of SEQ ID NO: 1 having no more than 5 amino acid substitutions, and a LCVR comprising an amino acid sequence of SEQ ID NO: 2 having no more than 2 amino acid substitutions.

### Pharmaceutical Compositions and Administration

The present disclosure provides therapeutic pharmaceutical compositions comprising the PD-1 inhibitors disclosed herein. Such pharmaceutical compositions may be formulated with suitable pharmaceutically acceptable carriers, excipients, buffers, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., “Compendium of excipients for parenteral formulations” PDA, J Pharm Sci Technol 52:238-311 (1998).

The dose of PD-1 inhibitor (e.g., anti-PD-1 antibody) may vary depending upon the age and the size of a subject to be administered, target disease, conditions, route of administration, and the like. When a PD-1 inhibitor of the present disclosure is used for treating or inhibiting the growth of liver cancer, lung cancer, or head and neck cancer, it may be advantageous to administer the PD-1 inhibitor at a single dose of about 0.1 to about 100 mg/kg body weight. Depending on the severity of the condition, the frequency and the duration of the treatment can be adjusted. In certain embodiments, the PD-1 inhibitor of the present disclosure can be administered as an initial dose of at least about 0.1 mg to about 800 mg, about 1 to about 1000 mg, about 1 to about 800 mg, about 5 to about 500 mg, or about 10 to about 400 mg. In certain embodiments, the initial dose may be followed by administration of a second or a plurality of subsequent doses of the PD-1 inhibitor in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. The pharmaceutical composition can be also delivered in a vesicle, in particular a liposome (see, e.g., Langer (1990) Science 249:1527-1533).

The use of nanoparticles to deliver the PD-1 inhibitor of the present disclosure is also contemplated herein. Antibody-conjugated nanoparticles may be used both for therapeutic and diagnostic applications. Antibody-conjugated nanoparticles and methods of preparation and use are described in detail by Arruebo et al., 2009, “Antibody-conjugated nanoparticles for biomedical applications,” J. Nanomat., Vol. 2009, Article ID 439389, 24 pages. Nanoparticles may be developed and conjugated to antibodies contained in pharmaceutical compositions to target cells. Nanoparticles for drug delivery have also been described in, for example, U.S. Pat. No. 8,257,740 or U.S. Pat. No. 8,246,995.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, and intramuscular injections, drip infusions, etc. These injectable preparations may be prepared by methods publicly known.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 1000 mg per dosage form in a unit dose, such as about 5 to about 600 mg, about 5 to about 350 mg, or about 10 to about 300 mg.

In certain embodiments, the present disclosure provides a pharmaceutical composition or formulation comprising a therapeutic amount of a PD-1 inhibitor (e.g., cemplimab or a bioequivalent thereof) and a pharmaceutically acceptable carrier. Non-limiting examples of pharmaceutical compositions comprising an anti-PD-1 antibody provided herein that can be used in the context of the present disclosure are disclosed in US 2019/0040137.

The present disclosure also provides kits comprising a PD-1 inhibitor (e.g., cemplimab or a bioequivalent thereof) for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for treating a patient afflicted with liver cancer, lung cancer, or head and neck cancer, the kit comprising: (a) a therapeutically effective dosage of a PD-1 inhibitor (e.g., cemplimab or a bioequivalent thereof); and (b) instructions for using the PD-1 inhibitor in any of the methods disclosed herein.

### Administration Regimens

In certain embodiments, the methods disclosed herein include administering to the tumor of a subject in need thereof a therapeutically effective amount of a PD-1 inhibitor (e.g., cemiplimab or a bioequivalent thereof) in multiple doses, e.g., as part of a specific therapeutic dosing regimen. For example, the therapeutic dosing regimen may comprise administering one or more doses of a PD-1 inhibitor to the subject at a frequency of about once a day, once every two days, once every three days, once every four days, once every five days, once every six days, once a week, once every two weeks, once every three weeks, once every four weeks, once every five weeks, once every six weeks, once every eight weeks, once every twelve weeks, once a month, once every two months, once every three months, once every four months, twice a day, twice every two days, twice every three days, twice every four days, twice every five days, twice every six days, twice a week, twice every two weeks, twice every three weeks, twice every four weeks, twice every five weeks, twice every six weeks, twice every eight weeks, twice every twelve weeks, twice a month, twice every two months, twice every three months, twice every four months, three times a day, three times every two days, three times every three days, three times every four days, three times every five days, three times every six days, three times a week, three times every two weeks, three times every three weeks, three times every four weeks, three times every five weeks, three times every six weeks, three times every eight weeks, three times every twelve weeks, three times a month, three times every two months, three times every three months, three times every four months or less frequently or as needed so long as a therapeutic response is achieved. In one embodiment, one or more doses of the PD-1 inhibitor are administered as a neoadjuvant once every three weeks. In one embodiment, one or more doses of the PD-1 inhibitor are administered as a post-surgery adjuvant once every three weeks.

In certain embodiments, the one or more doses are administered in at least one treatment cycle—e.g., 1, 2, 3, 4, 5, 6, 7, 8, 9, or 10 treatment cycles. The methods, according to this aspect, comprise administering to a subject in need thereof at least one neoadjuvant treatment cycle, and optionally at least one adjuvant treatment cycle, each treatment cycle comprising administration of 1, 2, 3, 4, 5, 6, 7, 8, 9, 10 or more doses of a PD-1 inhibitor (e.g., cemplimab or a bioequivalent thereof). In certain embodiments, each dose of the PD-1 inhibitor comprises 0.1, 1, 0.3, 3, 4, 5, 6, 7, 8, 9 or 10 mg/kg of the patient's body weight. In certain embodiments, each dose comprises 5-1000 mg of the PD-1 inhibitor, for example 5, 10, 15, 20, 25, 40, 45, 50, 60, 70, 80, 90, 100, 150, 200, 250, 300, 350, 400, 450, 500, 550, 600, 650, 700, 750, 800, 850, 900, 950 mg or more of the PD-1 inhibitor. In some embodiments, the PD-1 inhibitor is administered in 2 neoadjuvant treatment cycles. In some embodiments, the PD-inhibitor is further administered in 8 post-surgery adjuvant treatment cycles. In some embodiments, neoadjuvant treatment comprises 2 treatment cycles, each cycle comprising 1 dose (e.g., 350 mg Q3W) of the PD-1 inhibitor. In some embodiments, adjuvant treatment comprises 8 treatment cycles, each cycle comprising 1 dose (e.g., 350 mg Q3W) of the PD-1 inhibitor.

### Dosage

The amount of PD-1 inhibitor (e.g., cemiplimab or a bioequivalent thereof) administered to a subject according to the methods disclosed herein is, generally, a therapeutically effective amount. As used herein, the term “therapeutically effective amount” means an amount of a PD-1 inhibitor administered as a neoadjuvant prior to planned surgery for treating liver cancer, lung cancer, or head and neck cancer that results in one or more of: (a) inhibition of tumor growth, or an increase in tumor necrosis, tumor shrinkage and/or tumor disappearance; (b) a reduction in the severity or duration of a symptom or an indication of the cancer—e.g., a tumor lesion; (c) delay in tumor growth and development; (d) inhibition of tumor metastasis; (e) prevention of recurrence of tumor growth; (f) increase in survival of a subject with the cancer; and/or (g) delay of surgery, each as compared to an untreated subject or a subject treated with surgical resection alone.

In certain embodiments, a therapeutically effective amount of the PD-1 inhibitor (e.g., cemplimab or a bioequivalent thereof) can be from about 0.05 mg to about 1000 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg.

The amount of a PD-1 inhibitor (e.g., cemplimab or a bioequivalent thereof) contained within an individual dose may be expressed in terms of milligrams of antibody per kilogram of subject body weight (i.e., mg/kg). In certain embodiments, the PD-1 inhibitor used in the methods disclosed herein may be administered to a subject at a dose of about 0.0001 to about 100 mg/kg of subject body weight. In certain embodiments, an anti-PD-1 antibody may be administered at dose of about 0.1 mg/kg to about 20 mg/kg of a patient's body weight. In certain embodiments, the methods of the present disclosure comprise administration of a PD-1 inhibitor (e.g., an anti-PD-1 antibody) at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In certain embodiments, an individual dose amount of a PD-1 inhibitor (e.g., cemplimab or a bioequivalent thereof) administered to a patient may be less than a therapeutically effective amount, i.e., a subtherapeutic dose. For example, if the therapeutically effective amount of a PD-1 inhibitor comprises 3 mg/kg, a subtherapeutic dose comprises an amount less than 3 mg/kg, e.g., 2 mg/kg, 1.5 mg/kg, 1 mg/kg, 0.5 mg/kg or 0.3 mg/kg. As defined herein, a “subtherapeutic dose” refers to an amount of the PD-1 inhibitor that does not lead to a therapeutic effect by itself. However, in certain embodiments, multiple subtherapeutic doses of a PD-1 inhibitor are administered to collectively achieve a therapeutic effect in the subject.

In certain embodiments, each dose comprises 0.1-10 mg/kg (e.g., 0.3 mg/kg, 1 mg/kg, 3 mg/kg, or 10 mg/kg) of PD-1 inhibitor (e.g., cemplimab or a bioequivalent thereof) based on the subject's body weight. In certain other embodiments, each dose comprises 5 to 600 mg of the PD-1 inhibitor, e.g., 5 mg, 10 mg, 15 mg, 20 mg, 25 mg, 30 mg, 40 mg, 45 mg, 50 mg, 100 mg, 150 mg, 200 mg, 250 mg, 300 mg, 400 mg, 500 mg, 600 mg, 700 mg, 800 mg, 900 mg, or 1000 mg of the PD-1 inhibitor.

In one embodiment, a therapeutically effective amount of PD-1 inhibitor (e.g., cemiplimab or a bioequivalent thereof) is 350 mg intravenously administered as neoadjuvant treatment prior to planned surgery for liver cancer, lung cancer, or head and neck cancer. In some embodiments, another therapeutically effective amount of PD-1 inhibitor (e.g., cemiplimab or a bioequivalent thereof) is 350 mg intravenously administered as an adjuvant treatment after surgery.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Clinical Trial of Neoadjuvant Cemiplimab for the Treatment of Resectable NSCLC, HCC, and HNSCC

This study is a phase 2a, multi-cohort study of neoadjuvant cemiplimab for the treatment of resectable non-small cell lung cancer (NSCLC), hepatocellular carcinoma (HCC), and head and neck squamous cell carcinoma (HNSCC), and neoadjuvant cemiplimab with or without chemotherapy for NSCLC. Cemiplimab is a fully human monoclonal anti-PD-1 antibody comprising a heavy chain having the amino acid sequence of SEQ ID NO: 9 and a light chain having the amino acid sequence of SEQ ID NO: 10; an HCVR/LCVR amino acid sequence pair comprising SEQ ID NOs: 1/2; and heavy and light chain CDR sequences comprising SEQ ID NOs: 3-8, as described herein. See also U.S. Pat. No. 9,987,500. The study includes the following cohorts: A1, A2, A3 (patients with resectable NSCLC); B (patients with resectable HCC); and C (patients with resectable HNSCC).

Study objectives: One objective of this study is to evaluate the clinical activity of neoadjuvant cemiplimab therapy in patients with resectable NSCLC, HCC, and HNSCC lesions, as measured by pathological evaluations of resected tumors. For Cohorts A1, A2, A3 (NSCLC), a primary objective is to evaluate major pathological response (MPR). For Cohort B (HCC), a primary objective is to evaluate significant tumor necrosis (STN). For Cohort C (HNSCC), a primary objective is to evaluate major treatment effect (MTE). Additional objectives of this study include: assessing the anti-tumor activity of neoadjuvant and adjuvant cemiplimab therapy as defined by multiple criteria, determining the safety and tolerability of neoadjuvant and adjuvant cemiplimab therapy including delay to surgery, and assessing the change in tumor-infiltrating CD8 T-cell density and exploring correlation to the pathological response to therapy.

Rationale: While the biology of each cohort is distinct, there is significant rationale for the inclusion of multiple, histologic solid tumor types in the design of this trial. For one, there are defined carcinogens for the three malignancies on which this study focuses (smoking in NSCLC; viral [HBV, HCV] infection, alcohol, and fatty liver disease in HCC; and viral [HPV] infection, smoking, and alcohol in HNSCC), that will allow comparative future studies as a result of the current studies. Alongside potential viral antigens, these tumor types carry a moderate to high mutational burden (Alexandrov et al., Nature, 2013; 500(7463):415-421) and should thus have a reasonable number of neoantigens that can be recognized by the adaptive immune system. There is also a high prevalence of risk factors associated with a higher incidence of these cancers, such as cigarette smoking. PD-1 blockade has been approved in the metastatic setting in all three tumor types—with a safety profile far superior to standard chemotherapy approaches-demonstrating they can be responsive to immunotherapy even though some 70-85% of patients still do not experience clinical benefit, defined as PR or better (Antonia et al., N Engl J Med 2017; 377:1919-1929; El-Khoueiry et al., Lancet. 2017; 389(10088):2492-2502; Bauml et al., Journal of Clinical Oncology: Official Journal of the American Society of Clinical Oncology. 2017:Jco2016701524; Borghaei et al., N Engl J Med. 2015; 373(17):1627-1639; Fehrenbacher et al., Lancet. 2016; 387(10030):1837-1846; Garon et al., N Engl J Med. 2015; 372(21):2018-2028).

Cancers are increasingly being detected at an early time point due to screening, when patients are still candidates for surgical resection. However, there is a significant, clinical need for novel treatments for these cancers given high rates of locoregional or distant recurrence, presumably due to micrometastatic disease insensitive to cytotoxic therapy. Neoadjuvant and adjuvant chemotherapy, or targeted therapy, have never demonstrated a clinical benefit in HCC, while patients with locally advanced NSCLC and HNSCC typically receive chemotherapy and/or radiation therapy (RT), although the survival benefit is modest and toxicity significant, particularly in NSCLC, where the survival benefit of neoadjuvant or adjuvant chemotherapy is a mere 5%. Hence, there remains a serious clinical need.

As with virtually all cancer therapies, immunotherapy was initially evaluated in the metastatic setting; however, it is hypothesized that patients with locoregional/resectable disease may benefit even more than patients with metastatic disease due to greater immune fitness, reduced tumor heterogeneity, and more localized disease.

The pathological response variables and thresholds selected for the current study are based upon pathological response assessments validated in neoadjuvant studies utilizing chemotherapy and/or other treatment modalities (Hellman et al., The Lancet Oncology. 2014; 15(1): e42-50; Pataer et al., J Thorac Oncol. 2012; 7(5):825-832; Allard et al., J Hepatol. 2015; 63(1):83-92). Less is understood regarding the translation of these criteria to the neoadjuvant setting in the context of immunotherapy. The possibility exists that additional or distinct variables or thresholds may be more pertinent in understanding the impact and benefit of immunotherapy in a neoadjuvant setting, which will be explored through the course of this study.

Study endpoints: The primary endpoint of this study is the efficacy of neoadjuvant therapy in patients with resectable NSCLC, HCC, and HNSCC lesions, defined as follows: MPR defined as ≤10% viable tumor within resection, at time of surgery is the primary endpoint for the NSCLC Cohorts A1, A2 and A3; STN defined as >70% necrosis of the tumor, based on pathologic analysis of gross tumor resection, at time of surgery is the primary endpoint for the HCC Cohort B; and MTE defined as tumor necrosis and/or giant cell/histiocytic reaction to keratinous debris in >70% of the pre-treatment tumor area, at time of surgery is the primary endpoint for the HNSCC Cohort C.

Other endpoints include: delay to surgery defined as surgery >28 days following the end of the cycle of last dose of cemiplimab (chemotherapy for cohort A3) in neoadjuvant period; disease-free survival (DFS) defined as the time from date of surgery until recurrence of tumor or death from any cause after successful surgery and recovery; overall response rate (ORR) defined as the percent of patients with a complete response (CR; 100% tumor necrosis) or partial response (PR; ≥30% decrease in tumor size) (patients who are not evaluable for response will be considered as non-responders); overall survival (OS) defined as the time from the first dosing of cemiplimab (chemotherapy for cohort A3) and date of death for any reason; OS rate at 12 months, 18, 24, 36, 48, and 60 months; incidence of treatment-emergent adverse events (TEAEs) (including perioperative complications), immune-related adverse events (irAEs), serious adverse events (SAEs), deaths, laboratory abnormalities (Grade 3 or higher per Common Terminology Criteria for Adverse Events); change in tumor-infiltrating CD8 T-cell density, defined as the change from baseline to the time of surgery.

Study variables: Baseline characteristics will include standard demography (e.g., age, race, weight, height, etc.), disease characteristics including medical history, and medication history for each patient. Efficacy variables include pathological evaluation of resected tumors. For NSCLC: MPR defined as ≤10% viable tumor within resection. MPR is a surrogate for clinical benefit developed and validated with previous NSCLC, neoadjuvant chemotherapy studies (Hellman et al., The Lancet Oncology. 2014; 15(1): e42-50; Pataer et al., J Thorac Oncol. 2012; 7(5):825-832). For HCC: STN defined as >70% necrosis of the tumor, based on pathologic analysis of gross tumor resection. Tumor necrosis of >70% of tumor has been shown in HCC to correlate with clinical outcome (Allard et al., J Hepatol. 2015; 63(1):83-92). For HNSCC: MTE is defined as tumor necrosis and/or giant cell/histiocytic reaction to keratinous debris in >70% of the pre-treatment tumor area. Other efficacy variables include: DFS, ORR, OS, change in tumor-infiltrating CD8 T-cell density.

Study design: Eligible patients with a known diagnosis of resectable NSCLC, HCC, or HNSCC will be enrolled in the following cohorts:

Cohort A1: Cohort A1 will enroll approximately 21 NSCLC patients to receive 350 mg cemiplimab 350 mg every 3 weeks (Q3W)×2 cycles in the neoadjuvant setting, followed by adjuvant therapy with 8 cycles of cemiplimab therapy along with 4 cycles of standard platinum-doublet chemotherapy.

Cohort A2: Cohort A2 will enroll approximately 21 NSCLC patients to receive 2 cycles of neoadjuvant cemiplimab 350 mg IV Q3W and 2 cycles of adjuvant platinum-doublet chemotherapy, followed by 8 additional cycles of adjuvant cemiplimab 350 mg IV Q3W monotherapy plus 2 cycles of platinum-doublet chemotherapy. All patients will receive a total of 4 cycles of split standard platinum-doublet chemotherapy, 2 in the neoadjuvant setting and 2 following surgery.

Cohort A3: Cohort A3 will enroll approximately 10 NSCLC patients to receive 2 cycles of neoadjuvant platinum-doublet chemotherapy before surgery, 2 additional cycles of adjuvant platinum-doublet chemotherapy followed by 8 additional cycles of adjuvant cemiplimab 350 mg IV Q3W. All patients will receive a total of 4 cycles of split, standard platinum-doublet chemotherapy, 2 in the neoadjuvant setting and 2 following surgery.

Cohort B: Cohort B will enroll approximately 21 HCC patients to receive neoadjuvant cemiplimab 350 mg IV Q3W for 2 cycles before surgery. In the adjuvant setting, patients will be administered 8 cycles of cemiplimab 350 mg IV Q3W

Cohort C: Cohort C will enroll approximately 21 HNSCC patients to receive neoadjuvant cemiplimab 350 mg IV Q3W for 2 cycles before surgery. Following surgery, patients will receive standard adjuvant chemotherapy and/or radiation. After the standard adjuvant therapy, patients will receive 8 cycles of adjuvant cemiplimab treatment Q3W.

Neoadjuvant therapy: Patients enrolled in cohorts A1, A2, B, and C will receive 2 doses of cemiplimab 350 mg IV Q3W before surgery. Patients will be observed for 1 hour following administration of cemiplimab, with vital signs monitored at the initiation of the infusion and completion of the infusion. The target administration is 2 doses, dosed 21 days apart before the time of surgery. Patients in cohort A2 will receive platinum-doublet chemotherapy the same day as cemiplimab is administered. Patients in exploratory cohort A3 will receive standard platinum-doublet on the Q3W dosing schedule without neoadjuvant cemiplimab. PD-L1 mRNA expression was plotted for squamous and non-squamous liver cancer tumors in The Cancer Genome Atlas (TCGA). Transcripts per Million (TPM) of liver tumors were plotted using OmicSoft ArrayStudio software, version 10.0.1.50. PD-L1 mRNA expression results are in whole based upon data generated by the TCGA Research Network (https://www.cancer.gov/tcga).

Neoadjuvant therapy for NSCLC Cohort: Patients with NSCLC enrolling into this trial will be enrolled into cohorts A1, A2, and A3 in a 2:2:1 randomization fashion. Cohort A3 will enroll only approximately 10 patients to allow for comparison of exploratory endpoints; this cohort receives standard therapy during the neoadjuvant period. Patients in this cohort receive 4 cycles of platinum-based chemotherapy, typically consisting of cisplatin or carboplatin in combination with pemetrexed (for non-squamous tumors) or paclitaxel (for squamous cell carcinoma). All patients enrolling in cohort A2 or cohort A3 will receive a total of 4 cycles of split, standard chemotherapy, 2 in the neoadjuvant setting and 2 following surgery. Cohort A2 will receive 2 cycles of neoadjuvant and 2 cycles of adjuvant combination chemo-immunotherapy, followed by 6 additional cycles of cemiplimab monotherapy. Cohort A3 will receive only neoadjuvant chemotherapy, but this group will receive 2 additional cycles of adjuvant chemotherapy alongside 8 cycles of adjuvant cemiplimab following surgery (to ensure potential benefit over standard of care for all trial patients).

Surgery after neoadjuvant therapy: The study design calls for 2 doses of cemiplimab and/or platinum-doublet chemotherapy to be administered during the neoadjuvant period 21 days apart. Surgery should generally be scheduled for 4-6 weeks following the first dose of neoadjuvant therapy. If a patient's tumor morbidity does not allow for delayed surgery long enough to receive their second dose of cemiplimab they may proceed to surgery as early as 14 days following their first dose of cemiplimab (applies to chemotherapy for cohort A3). For patients who receive 2 cycles of planned cemiplimab, surgery should occur at least 1 day after the second dose of cemiplimab, and for patients receiving chemotherapy, upon blood-count recovery from the most recent cycle of chemotherapy. Given the relatively small benefit derived from neoadjuvant/adjuvant chemotherapy alone in patients with NSCLC, and the high likelihood of recurrence in these patients and in HCC patients who will receive no chemotherapy or radiation, all NSCLC and HCC patients will be given 8 additional cycles of cemiplimab, Q3W, following recovery from surgery. All HNSCC patients will receive the same 8 additional cycles of cemiplimab following completion of standard-of-care radiation with or without chemotherapy.

Adjuvant therapy: Patients in cohorts A1, A2, A3, and B will receive 8 cycles of adjuvant cemiplimab 350 mg IV Q3W, following recovery from surgery. Patients in cohort C will receive 8 cycles of adjuvant cemiplimab, Q3W, following completion of standard-of-care adjuvant radiation with or without chemotherapy. Patients will be followed with regular surveillance regardless of whether they receive standard or experimental adjuvant therapy. Patients with incomplete surgical resection will not receive adjuvant therapy with cemiplimab but will be managed per standard of care for their residual disease. Patients with tumor recurrence during the adjuvant therapy phase will discontinue further cemiplimab and will be managed per standard of care for their recurrent disease.

Adjuvant therapy for NSCLC Cohort: All NSCLC patients will receive 8 additional cycles of cemiplimab in the adjuvant setting upon recovery from surgery. Cohort A1 will receive 4 cycles of standard, platinum-doublet chemotherapy with the first 4 of the 8 additional cycles of cemiplimab. Cohorts A2 and A3 will receive 2 additional cycles of platinum-doublet chemotherapy with the first 2 of these 8 additional cycles of cemiplimab. The first dose of combination chemotherapy and cemiplimab will be scheduled at within 8 weeks following the surgery, and adjuvant therapy will be given on a standard Q3W schedule; however, scheduling changes are permitted at the discretion of the treating physician.

Adjuvant therapy for HCC Cohort: Given the dearth of adjuvant or neoadjuvant options for HCC patients, and the high likelihood of recurrence, all patients will be given 8 additional cycles of cemiplimab, infused Q3W. The first dose will be scheduled within 8 weeks following the surgery.

Adjuvant therapy for HNSCC Cohort: Following surgery, patients will receive standard adjuvant radiation with or without chemotherapy as per standard of care (SOC). Given the paucity of data of combined chemotherapy or radiotherapy and PD-1 blockade in HNSCC patients, no cemiplimab will be given during SOC adjuvant therapy; however, these patients will receive 8 cycles of adjuvant cemiplimab following completion of standard adjuvant therapy. The first dose of cemiplimab will be administered within 8 weeks following SOC adjuvant therapy.

Follow-up: During the post-surgical period, patients will be evaluated in surgery follow up every 4 weeks until the initiation of adjuvant therapy to assess for adverse events. All patients should be evaluated for 90 days after their final dose of cemiplimab for adverse events. Imaging in the post-surgical period should be performed every 12 weeks from surgery for the first 2 years, and thereafter per standard of care for up to 5 years following surgery. During this 5 year period, the disease status of patients will be assessed every 12 weeks from surgery, and upon disease recurrence, patients will be monitored for survival every 12 weeks from the date of recurrence. If a patient does not report for in-person study visits at any time during the post-surgical period, they should continue to be followed for survival every 12 weeks from the date of last contact, through chart review or by telephone until death, withdrawal of consent, or the end of the study, whichever occurs earlier.

Study population: Approximately 94 patients with a known diagnosis of resectable NSCLC, HCC, or HNSCC will be enrolled.

Inclusion Criteria: A patient must meet all of the following criteria to be eligible for inclusion in the study: (1) Men and women of age ≥18 years; (2) Patient must have a known diagnosis of NSCLC, HCC, or HNSCC; histological diagnosis of NSCLC and HNSCC is required; pre-treatment and diagnostic biopsies can be done simultaneously if the imaging clearly supports the required diagnosis (e.g., HCC); (a) NSCLC: Patients will have either a nodal involvement or primary tumor ≥4 cm; (b) HCC: Initial diagnosis of HCC may be made using radiographic parameters; however, pre-treatment, core needle biopsies are mandatory for all cohorts; (c) HNSCC: Patients will have primary tumor site of oral cavity, oropharynx, larynx, or hypopharynx; (3) Patient must be willing and able to provide blood samples (up to 120 mL at certain visits) at the time points indicated; (4) Patient must be willing and able to have excisional or core needle biopsies of tumor prior to initiation of cemiplimab (chemotherapy for cohort A3) (goal is up to 4 biopsies, final number to be determined by the surgeon and radiologist performing the procedure as safe. Patients receiving anti-coagulation or anti-platelet therapy must be candidates for safe interruption of this therapy prior to biopsy, and coagulation parameters (aPTT/INR) must have normalized at the time of biopsy to ≤1.5 ULN. For HCC lesions, the biopsy must be performed with imaging guidance, and the biopsy needled must first traverse at least 1 cm of normal hepatic parenchyma in order to mitigate potential bleeding complications; (5) Eastern Cooperative Oncology Group (ECOG) performance status of 0 or 1. The exception will be patients carrying long-term disability (such as cerebral palsy) where the disability is neither acute nor progressive, and unlikely to significantly affect their response to therapy; (6) Patient is determined to be a surgical candidate for resection of their tumor; (7) Patient is able to understand and willing to sign a written informed consent as specified by health authorities and institutional guidelines; (8) Adequate organ and bone marrow function; and (9) Willing and able to comply with clinic visits and study-related procedures and requirements.

Exclusion Criteria: A patient who meets any of the following criteria will be excluded from the study: (1) Patients who have had any systemic anti-cancer therapy or radiotherapy within 6 months prior to entering the study for their current tumor or a different primary tumor; (2) Patients whose tumor burden or pace of tumor growth will not permit delaying surgery through 2 doses of neoadjuvant cemiplimab (chemotherapy for Cohort A3); (3) Patients who have participated in a study of an investigational agent or an investigational device within 4 weeks of study therapy or 5 half-lives (whichever is longer); (4) Patients who have had major surgery within 14 days prior to initiation of neoadjuvant therapy; (5) Patients with metastatic disease, for whom the intent of surgery would not be curative; (6) Uncontrolled, intercurrent illness including, but not limited to: ongoing or active infection requiring antibiotics (exception is a brief (510 days) course of antibiotics to be completed before initiation of treatment), symptomatic congestive heart failure, unstable angina pectoris, or psychiatric illness/social situations that would limit compliance with study requirements; (7) Is receiving systemic steroid therapy or any other form of immunosuppressive therapy within 7 days prior to the first dose of study treatment. Patients on chronic steroids (more than 4 weeks at stable dose) equivalent to ≤10 prednisone will not be excluded; (8) Has active autoimmune disease that has required systemic treatment in the past 1 year (i.e., with use of disease-modifying agents, corticosteroids, or immunosuppressive drugs). Replacement therapy (e.g., thyroxine, insulin, or physiologic, corticosteroid replacement therapy for adrenal or pituitary insufficiency, etc.) is acceptable; (9) Has a known, additional malignancy that is progressing and/or requires active treatment. Exceptions include patients with: basal cell carcinoma of the skin or squamous cell carcinoma of the skin that has undergone potentially curative therapy; in situ cervical or anal cancer; prostate cancer on stable dose of hormonal therapy without rising PSA; breast cancer who have been treated with curative intent, who may be on hormonal therapy; (10) Encephalitis, meningitis, or uncontrolled seizures in the year prior to informed consent; (11) History of interstitial lung disease (e.g., idiopathic pulmonary fibrosis, organizing pneumonia) or active, noninfectious pneumonitis that required immune-suppressive doses of glucocorticoids to assist with management. A history of radiation pneumonitis in the radiation field is permitted as long as pneumonitis resolved: 6 months prior to study treatment; (12) Uncontrolled infection with human immunodeficiency virus (HIV), HBV or hepatitis C infection (HCV); or diagnosis of immunodeficiency; (a) Patients will be tested for hepatitis C virus (HCV) and hepatitis B virus (HBV) at screening; (b) Patients with known HIV infection who have controlled infection (undetectable viral load (HIV RNA PCR) and CD4 count above 350 either spontaneously or on a stable anti-viral regimen) are permitted. For patients with controlled HIV infection, monitoring will be performed per local standards; (c) Patients with hepatitis B (HepBsAg+) who have controlled infection (serum HBV DNA PCR that is <100 IU/ml AND receiving anti-viral therapy for hepatitis B) are permitted. Patients with controlled infections must undergo periodic monitoring of HBV DNA. Patients must remain on anti-viral therapy for at least 6 months beyond the last dose of investigational study drug; (d) Patients who are hepatitis C virus antibody positive (HCV Ab+) who have controlled infection (undetectable HCV RNA by PCR either spontaneously or in response to a successful prior course of anti-HCV therapy) are permitted; (13) Receipt of a live vaccine within 28 days of planned start of study medication; (14) Prior allogeneic stem cell transplantation, or autologous stem cell transplantation; (15) Recipients of organ transplants; (16) Any medical co-morbidity, physical examination finding, or metabolic dysfunction, or clinical laboratory abnormality that renders the patient unsuitable for participation in a clinical trial due to high safety risks and/or potential to affect interpretation of results of the study; (17) Member of the clinical site study team or his/her immediate family; (18) Documented allergic or hypersensitivity response to any protein therapeutics (eg, recombinant proteins, vaccines, IV immune globulins, monoclonal antibodies, receptor traps, excipients of cemiplimab); (19) Known psychiatric or substance abuse disorders that would interfere with fulfilling the requirements of the study; (20) Women with a positive serum hCG pregnancy test at the screening/baseline visit. If positive, pregnancy must be ruled out by ultrasound for patient to be eligible; (21) Breastfeeding women are excluded; (22) Women of childbearing potential (WOCBP) or sexually active men whose partners are WOCBP, who are unwilling to practice highly effective contraception prior to the initial dose/start of the first treatment, during the study, and for at least 6 months after the last dose.

Study treatments: Patients enrolled in cohorts A1, A2, B, and C will receive 2 doses of cemiplimab (350 mg Q3W) IV before surgery. The target administration is 2 doses, dosed 21 days apart before the time of surgery. Patients in cohort A2 will receive platinum-doublet chemotherapy the same day as cemiplimab is administered. Patients in cohort A3 will receive standard platinum-doublet on the Q3W dosing schedule and no neoadjuvant cemiplimab. Patients in cohort C may receive standard-of-care radiotherapy with or without chemotherapy prior to receiving cemiplimab. Treatment and dosage information is summarized in Table 1 below:

TABLE 1
Regimen Descriptions

| | | | | | | Cycle |
|---|---|---|---|---|---|---|
| Agent | Cohorts | Dose | | Route | Schedule | Length |
| Cemiplimab | A1, A2, B, C | 350 | mg | IV | Days 1, 22. | 21 days |
| (neoadjuvant) | | | | | | |
| Cemiplimab | A1, A2, A3, B, C | 350 | mg | IV | Every 21 days for 8 | 21 days |
| (adjuvant) | | | | | cycles following surgery. | |
| Cisplatin | A1, A2, A3 | 75 | mg/m² | IV | Every 21 days, total | 21 days |
| | | | | | of 4 cycles. Timing | |
| | | | | | depends on cohort. | |
| Carboplatin | A1, A2, A3 (if deemed | AUC 5 | | IV | Every 21 days, total | 21 days |
| | cisplatin-ineligible) | | | | of 4 cycles. Timing | |
| | | | | | depends on cohort. | |
| Pemetrexed | A1, A2, A3 for | 500 | mg/m² | IV | Every 21 days, total | 21 days |
| | adenocarcinoma | | | | of 4 cycles. Timing | |
| | | | | | depends on cohort. | |
| Paclitaxel | A1, A2, A3 for | 175 | mg/m² | IV | Every 21 days, total | 21 days |

| | |
|---|---|
| squamous cell | of 4 cycles. Timing |
| carcinoma | depends on cohort. |

Concomitant medications and procedures: Any procedure performed or treatment administered of both prescription medications or over-the-counter preparations from the time of informed consent until 90 days after the last study treatment will be considered concomitant treatment. This includes medications and other therapies for which administration started since the informed consent form (ICF) had been signed and before the first dose of the study, and which will continue during the study, as well as any therapies started in the follow-up period to treat a study-drug-related adverse event (AE).

Prohibited medications and procedures: While participating in this study, a patient may not receive any treatment of a tumor other than those outlined herein, per the study's specified dosing regimens. Patients must not receive live vaccines during the study. Any other medication which is considered necessary for the patient's welfare, and which is not expected to interfere with the evaluation of the study drug, may be given with discretion. Patients using immunosuppressive doses (>10 mg per day of prednisone or equivalent) of systemic corticosteroids, other than for corticosteroid replacement, will not be eligible for the study.

Permitted medications and procedures: Standard antiemetics and preparative medications will be used for all patients receiving chemotherapy. Physiologic replacement doses of systemic corticosteroids are permitted, even if >10 mg/day prednisone equivalents. A brief course of corticosteroids for prophylaxis (eg, contrast dye allergy) or for treatment of non-autoimmune conditions (e.g., delayed-type hypersensitivity reaction caused by contact allergen) is permitted.

Statistical methods: For continuous variables, descriptive statistics will include the following information: the number of patients reflected in the calculation (n), mean, median, standard deviation, minimum, and maximum. For categorical or ordinal data, frequencies and percentages will be displayed for each category. For time-to-event data, Kaplan-Meier curves and estimate, and median survival rate at the key landmark timepoint along with 95% confidence interval (CI), will be provided. Primary efficacy analysis includes response rate, which will be summarized using descriptive statistics along with a two-sided Clooper-Pearson 95% CI calculated for each cohort. Secondary analysis of efficacy includes DFS, OS, ORR as measured by modified RECIST 1.1 (i.e., RECIST 1.1 (Eisenhauer, 2009) with no requirement for confirmation of response [PR/CR], as summarized in Tables 2 and 3 below).

TABLE 2
Response According to Modified RECIST 1.1 in
Patients with Target and Non-Target Lesions

| Target | | New | Overall |
|---|---|---|---|
| Lesions | Non-target Lesions | Lesions | Response* |
| CR | CR | No | CR |
| CR | Non-CR/non-PD | No | PR |
| CR | Not evaluated | No | PR |
| PR | Non-CR/non-PD/not all evaluated | No | PR |
| SD | Non-CR/non-PD/not all evaluated | No | SD |
| Not all | Non-PD | No | NE |
| evaluated | | | |
| PD | Any | Yes or no | PD |
| Any | PD* | Yes or no | PD |
| Any | Any | Yes | PD |

CR = complete response; PD = progressive disease; PR = partial response; SD = stable disease; NE = inevaluable.
*In exceptional circumstances, unequivocal progression in non-target lesions may be accepted as PD.

TABLE 3
Response According to Modified RECIST 1.1
in Patients with Non-Target Lesions Only

| Non-Target Lesions | New Lesions | Overall Response |
|---|---|---|
| CR | No | CR |
| Non-CR/non-PD | No | Non-CR/non-PD |
| Not all evaluated | No | NE |
| Unequivocal PD | Yes or No | PD |
| Any | Yes | PD |

### Example 2: Results of Clinical Trial of Neoadjuvant Cemiplimab for the Treatment of Resectable HCC (Cohort B)

This example provides results from the clinical trial of perioperative cemiplimab (anti-PD-1) for resectable HCC (NCT03916627, Cohort B), as described in Example 1. In this cohort, a total of 21 patients (Table 4) were enrolled.

TABLE 4
Patient demographics, baseline characteristics, and disposition

| Characteristic, n (%) | Resectable |
|---|---|
| unless stated otherwise | HCC (N = 21) |
| Median age, years (range) | 68 (45-82) |
| Male | 18 (85.7) |
| White | 9 (42.9) |
| ECOG performance status | |
| 0 | 18 (85.7) |
| 1 | 3 (14.3) |
| Median neoadjuvant treatment | 6 (5.3-6.7) |
| duration, weeks (range) | |

After initial imaging and biopsy, patients received 2 cycles of neoadjuvant cemiplimab (350 mg Q3W), followed by further imaging, after which the patients underwent surgical tumor resection and adjacent tissue sampling as soon as 23 days after starting treatment, and then an additional 8 adjuvant cemiplimab cycles (350 mg Q3W). Before treatment patients underwent 3D imaging with magnetic resonance imaging (MRI) and core needle biopsies of their tumor. Blood was collected for analysis throughout the perioperative screening period and prior to start of neoadjuvant treatment, and patients underwent repeat 3D MRI imaging immediately before surgical resection.

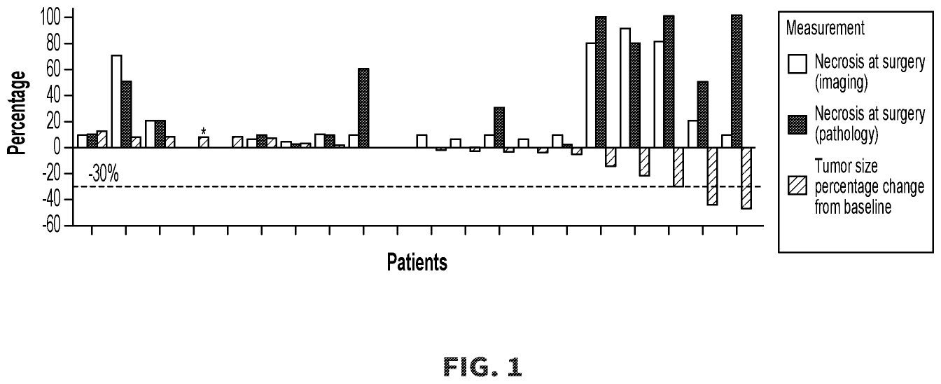

The primary endpoint was significant tumor necrosis, defined as >70% necrosis of the resected tumor based on pathologic analysis of gross tumor resection at the time of surgery. Secondary endpoints included delay of surgery, disease-free survival, overall response rate per modified RECIST 1.1, overall survival, adverse events (AEs), and change in lymphocyte infiltration. Patients underwent pre-treatment biopsies and regular blood collection throughout treatment to enable exploratory analyses including multiplex IHC and single-cell proteomic and transcriptomic analysis. As discussed herein, neoadjuvant therapy with cemiplimab suprisingly resulted in measurable, pathologic responses in HCC. Over the course of 18 months, all 21 patients received 2 cycles of preoperative cemiplimab, and all but 1 patient underwent successful resection; 1 patient was found to have metastatic disease at the time of surgery and resection was aborted. Tumor size change from baseline using RECIST criteria, together with measurement of tumor necrosis by perfusion analysis on MRI was assessed in each patient (FIG. 1).

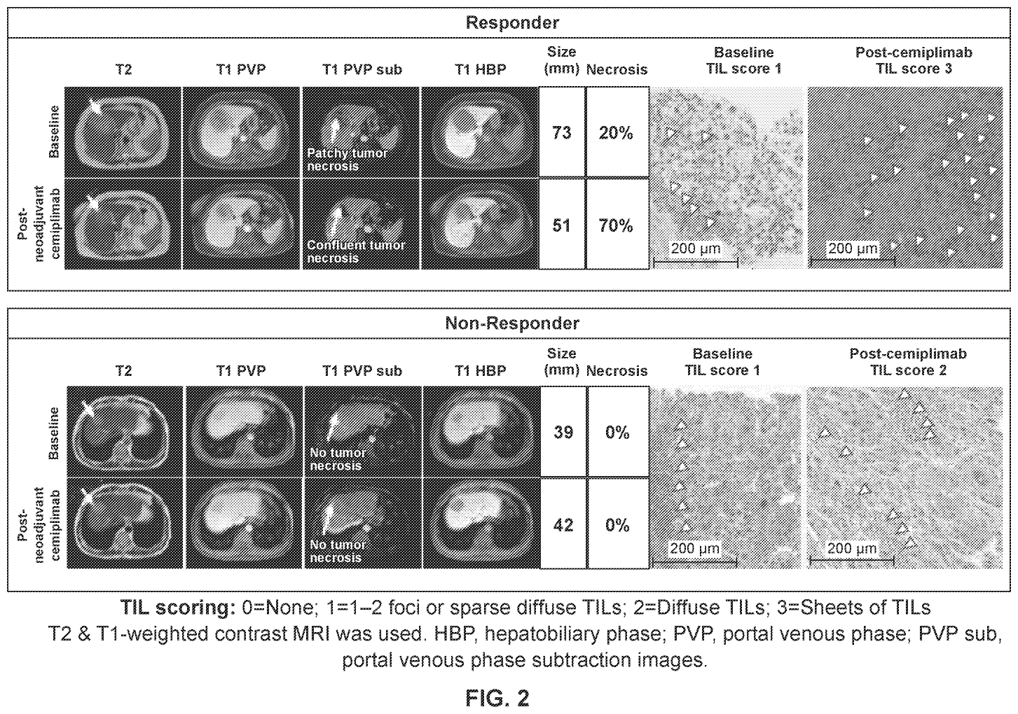

FIG. 2 shows representative MRI images in a responder and a non-responder with corresponding hematoxylin and eosin images demonstrating infiltration by immune cells, as quantified by tumor-infiltrating lymphocyte (TIL) score. 3D MRI was performed at the screening visit, surgery visit, and every 12 weeks in the post-surgical period for the first 2 years, then per standard of care for up to 5 years post-surgery.

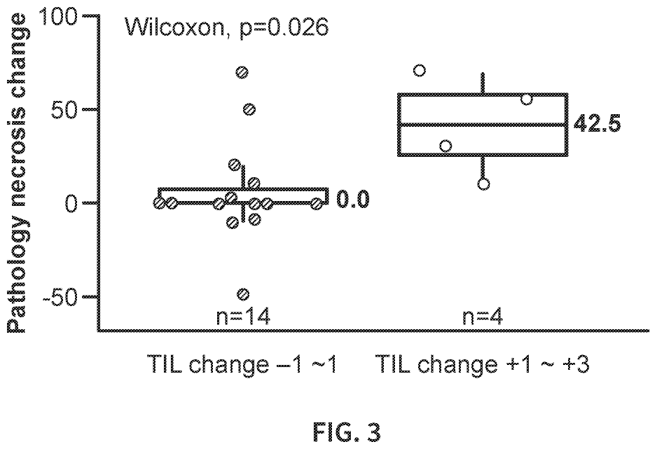

Pathological assessments of change in necrosis and tumor-infiltrating CD8 T-cell density from baseline was performed in pre-treatment biopsies and resected tumor samples, post-neoadjuvant treatment. Resected tumor samples were processed for tumor DNA, multiplex ion beam imaging, immunohistochemistry assays, mutational analysis, and single-cell biomarker analysis. Initial pathological assessment suggests an association between immune cell infiltration and tumor necrosis (FIG. 3). As shown in FIG. 3, there was a nominally significant difference in response (pathological necrosis change) between patients with increased TIL (TIL+2˜+3) versus patients with mild change in TIL (TIL change −1 ˜1) (p=0.026).

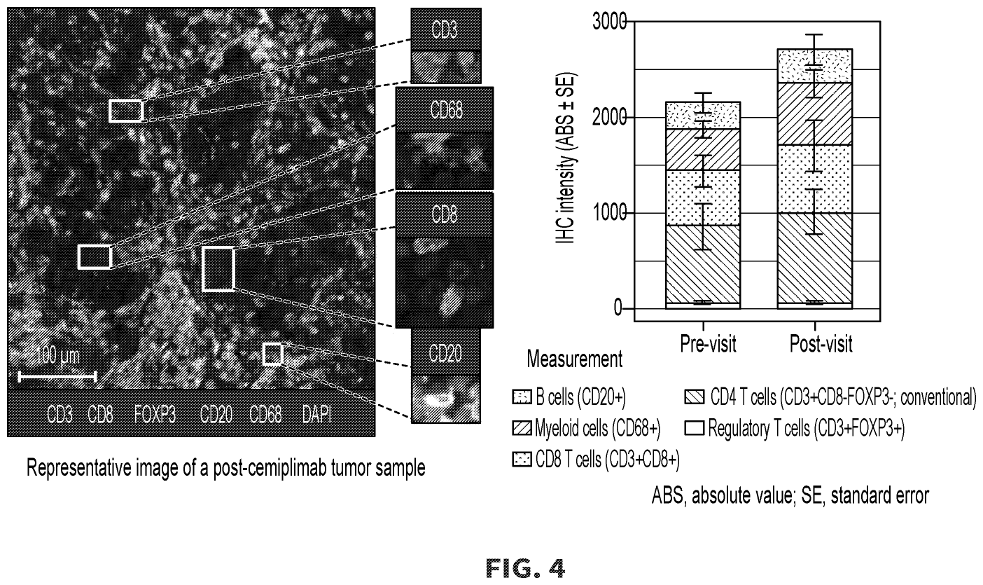

Tissue analysis is ongoing. Multiplex IHC describing myeloid and lymphoid infiltration patterns at baseline and following cemiplimab treatment are shown in FIG. 4.

The treatment was well tolerated: 19 (90.5%) of patients experienced at least one treatment-emergent AE (TEAE) of any grade, regardless of attribution (Table 5). The most common TEAEs of any grade were increased aspartate aminotransferase (n=6, 28.6%), increased alanine aminotransferase, increased blood creatine phosphokinase, constipation, and fatigue (each n=3, 14.3%). TEAEs were related to HCC but not related to study treatment. Grade ≥3 TEAE regardless of attribution occurred in six (28.6%) patients. Elevated blood creatine phosphokinase occurred in two patients (9.5%) and resolved without treatment. Treatment-related TEAEs of any grade occurred in six (28.6%) patients. One patient experienced Grade 3 pneumonitis during neoadjuvant therapy and surgery was delayed by 2 weeks per protocol defined surgical window. Cemiplimab treatment was not changed. Upon resolution of the event, successful surgical resection was performed.

TABLE 5
Summary of neoadjuvant TEAEs (any grade in ≥2 patients)

Total (N = 21)

| TEAE, n (%) | Any grade | Grade ≥3 |
|---|---|---|
| Any | 19 (90.5) | 6 (28.6) |
| Increased aspartate aminotransferase | 6 (28.6) | 1 (4.8) |
| Increased alanine aminotransferase | 3 (14.3) | 0 |
| Increased blood creatine phosphokinase | 3 (14.3) | 2 (9.5) |
| Constipation | 3 (14.3) | 0 |
| Fatigue | 3 (14.3) | 0 |
| Hypoalbuminemia | 2 (9.5) | 1 (4.8) |
| Abdominal pain | 2 (9.5) | 0 |
| Pruritis | 2 (9.5) | 0 |
| Rash | 2 (9.5) | 0 |
| Cough | 2 (9.5) | 0 |
| Infusion-related reaction | 2 (9.5) | 0 |
| Upper abdominal pain | 2 (9.5) | 0 |
| Hypocalcemia | 2 (9.5) | 0 |
| Anemia | 2 (9.5) | 0 |

Of the 20 patients with resected tumors, 7 (35%) had ≥50% tumor necrosis and 4 (20%) met the predefined endpoint of significant >70% tumor necrosis. Three (15%) of the 4 patients with >70% tumor necrosis had a pathological complete response. Similar patterns of change in degree of necrosis on pathological assessment and 3D MRI were observed at baseline and following completion of immunotherapy. Initial pathological assessment demonstrates a correlation between the presence of pre-existing tumor infiltrating lymphocytes and response. This is the largest trial reported to date of neoadjuvant PD-1 targeted monotherapy in HCC.

Cemiplimab demonstrated an acceptable safety-risk profile in patients with resectable HCC in neoadjuvant setting. The pathological response data support larger trials to identify optimal clinical endpoints that correlate with improvement in survival, and to establish the utility and safety of perioperative PD-1 blockade in patients with resectable HCC.

### Example 3: Neoadjuvant Cemiplimab Demonstrates Complete Pathologic Response in HCC

This example provides further results from the clinical trial of perioperative cemiplimab (anti-PD-1) for resectable HCC (NCT03916627, Cohort B), as described in Example 1. The study is a single-center, open-label, single-arm phase 2 trial in which cemiplimab monotherapy was administered before and after definitive surgery; 21 patients with early-stage HCC were enrolled, and all patients underwent surgery (Table 6).

TABLE 6
Patient demographics, baseline characteristics, and disposition

| Characteristic, n (%) | Resectable |
|---|---|
| unless stated otherwise | HCC (N = 21) |
| Age, median (range), years | 68 (45-82) |
| Male | 18 (85.7) |
| Asian | 11 (52.4) |
| ECOG performance status | |
| 0 | 18 (85.7) |
| 1 | 3 (14.3) |
| Neoadjuvant treatment duration, | 6 (5.3-6.7) |
| median (range), weeks | |
| Etiology of HCC, n (%) | |
| HBV | 8 (38.1) |
| HCV | 5 (23.1) |
| NASH/NAFLD | 5 (23.1) |
| ALD | 1 (4.8) |
| Fibrosis score, n (%) | |
| 0 | 1 (4.8) |
| 1 | 7 (33.3) |
| 2 | 4 (19.0) |
| 3 | 5 (23.8) |
| 4 | 4 (19.0) |
| Stage, n (%) | |
| Stage Ib | 6 (28.6) |
| Stage II | 14 (66.7) |
| Stage IIIB | 1 (4.8) |

ALD, alcoholic liver disease;
ECOG, European Cooperative Oncology Group;
HBV, hepatitis B virus;
HCC, hepatocellular carcinoma;
HCV, hepatitis C virus;
NAFLD, nonalcoholic fatty liver disease,
NASH, nonalcoholic steatohepatitis

Patients had to be 18 years or older and had confirmation of resectable HCC (Liver Imaging Reporting and Data System [LIRADS] score of 5 on imaging and/or biopsy-proven disease), an ECOG performance status of 0 or 1, and adequate liver function. Patients were enrolled regardless of underlying etiology of HCC; patients with a history of HCV or HBV were permitted if viral clearance had occurred or circulating virus was suppressed on HBV-directed therapies. Patients with HIV with an undetectable viral load by polymerase chain reaction (PCR) and a CD4+ T-cell count >350 cells/μL were permitted.

Patients were excluded from enrollment if they had metastatic disease, if the surgery was not expected to be curative, or if they had a known additional malignancy requiring active treatment. Patients could not be receiving chronic systemic immunosuppression or have active autoimmune disease requiring systemic treatment in the past year, except for patients with endocrinopathies on hormone replacement therapy. Pregnant women and transplant patients were excluded, as were any patients with a history of central nervous system or pulmonary inflammatory conditions.

Patients deemed surgical resection candidates were enrolled, underwent a core needle biopsy of their tumor (per protocol) under computed tomography (CT) guidance, and subsequently received two doses of neoadjuvant cemiplimab intravenous (IV) 350 mg every 3 weeks (Q3W). After the second dose of cemiplimab, patients underwent surgical resection. Gadoxetate-enhanced magnetic resonance imaging (MRI) was performed before initiation of treatment and again within 10 days prior to surgical resection, unless contraindicated, in which case patients underwent CT scans. Blood was collected at regular intervals and cryopreserved for later analysis. Upon recovery from surgery, patients received an additional eight cycles of cemiplimab IV 350 mg Q3W.

Tumor necrosis on pathologic examination was assessed by visually estimates of the percentage of necrosis seen within the resected tumor bed, as defined by the region within the tumor capsule delineated from normal hepatocytes, and in the pre-treatment biopsy. Necrosis in the biopsy was estimated based on the full core analysed; to measure the percentage of the tumor that was necrotic, the entire tumor bed was examined grossly for necrosis, then representative samples of the tumor (at least one section per cm of the largest dimension) were examined to confirm assessment; a complete pathologic response was defined as an absence of viable tumor in all sections analysed. The tumor-infiltrating lymphocytes (TILs) and tertiary lymphoid structure (TLS)-like aggregates were also quantified in these pre- and post-treatment specimens. The degree of tumor necrosis on preoperative MRI was defined as nonenhancing tissue on subtracted postcontrast T1-weighted images obtained during the portal venous phase; this necrosis quantification has previously been shown to correlate closely with degree of tumor necrosis on histopathologic assessment in HCC (Gordic et al., J Hepatol 2017; 67(6):1213-21).

Safety was monitored continuously throughout the trial: before surgery, postoperatively during adjuvant therapy, and for 90 days after the cessation of cemiplimab (monitoring in the adjuvant phase is ongoing). Safety and tolerability of neoadjuvant treatment through surgical resection is reported herein. AEs were assessed using the Common Terminology Criteria for Adverse Events (CTCAE, version 5.0).

The primary endpoint was significant tumor necrosis (STN), defined as >70% necrosis of resected tumor (Allard et al., J Hepatol 2015; 63(1600-0641 Electronic):83-92). Secondary endpoints included delay of surgery, defined as surgery >28 days following the second cycle of cemiplimab; overall response rate (ORR) (according to RECIST 1.1), defined as the percentage of patients with a complete response (CR; 100% tumor necrosis) or partial response (PR; ≥30% decrease in tumor size) documented by the investigator per RECIST 1.1 criteria. Also recorded was the percentage of patients with ≥50% tumor necrosis on pathologic examination of resected tumor; treatment emergent AEs (TEAEs), defined as AEs that were not present at baseline or represented the exacerbation of a pre-existing condition during the on-treatment period; immune-related AEs (IRAEs), defined as AEs that met protocol-defined immune-related criteria; and change in tumor-infiltrating CD8+ T-cell density, defined as the change from baseline to the time of surgery.

Tissue analysis: Pretreatment biopsies were formalin fixed and paraffin embedded (FFPE) for multiplex immunohistochemistry (mIHC) and immunofluorescence analysis, and additional biopsies were preserved in RNA-later for bulk RNA sequencing (BulkSeq) analysis (Remark et al., Sci Immunol, 2016; 1(1):aaf6925). A fully automated mIHC assay was performed on the Ventana Discovery ULTRA platform (Ventana Medical Systems, Tucson, AZ, USA) (Zhang et al., Lab Investig 2017; 97(7):873-85). Surgical tumor resections obtained after treatment were similarly preserved for analysis. The degree of tumor necrosis and the presence of TILs and TLS-like structures were quantified on FFPE pretreatment biopsies and post-treatment tumor resections. Following pathologic assessment, resected tumors were sampled to allow for paired BulkSeq on post-treatment tissue; in patients with remaining tissue, tumors and adjacent tissue were dissociated into a single-cell suspension, and this subset was analysed by mass cytometry using previously used methods and established panels.

BulkSeq was performed on pretreatment biopsies and resection samples that were stored in RNA-later. Sequencing and analysis were performed as previously described. To identify cell types within the BulkSeq data, previously described gene signatures for CD8+ T cells (Lei et al., Clinical Cancer Research 2021), naive, cytotoxic, or activated/dysfunctional lymphocytes (Van der Leun et al., Nat Rev Cancer, 2020; 20(4):218-32), or B cells and T regulatory cells (Szabo et al., Nat Commun 2019; 10(1):1-16) were used to quantify lymphocyte populations within tumor specimens pre- and post-treatment; the monocyte-derived macrophage population was defined using a gene signature that included CSF1R, CSF3R, CD163, CD68, C1QA, CD14, TFEC. The scores were then generated by taking the log of the total transcript count of all genes comprising the signature.

Statistical analysis: Primary efficacy outcomes were measured based on patients that completed surgery, secondary efficacy outcomes were measured in the full analysis set, and safety outcomes were assessed according to the safety analysis set. Rates of STN were summarized using frequencies and percentages, and two-sided 95% confidence intervals were calculated using the Clopper-Pearson method. The secondary efficacy endpoint, ORR, was measured by RECIST 1-1 criteria; confirmation of CR or PR was not feasible due to subsequent removal of tumor. All AEs reported in this study were coded using the currently available version of the Medical Dictionary for Regulatory Activities (MedDRA). Correlation between radiographic and pathologic estimations of necrosis and radiographic tumor shrinkage were evaluated by Spearman correlation, and nominal p values and correlation coefficients were reported. For cell subsets identified using gene signatures in BulkSeq data, Wilcoxon signed-rank test was used to assess significance between patient groups for these scores.

Results: All 21 patients enrolled in the trial underwent biopsies, and subsequently received two doses of cemiplimab. Most patients were Asian (52%), and the most common underlying etiology was HBV infection (Table 6). Twenty patients were stage Ib-II on the AJCC UICC 8th edition, and one patient was stage IIIb radiographically due to branch portal vein invasion. The median time from initiation of cemiplimab to surgical excision was 29 days, with one patient undergoing surgery as soon as 22 days after initiation of immunotherapy. One patient was found to have metastatic disease upon surgical exploration and resection was aborted.

Cemiplimab demonstrated an acceptable risk-benefit profile. Twenty (95%) patients experienced AEs of any grade during the neoadjuvant treatment period (Table 7). There were seven (33%) patients who experienced grade 3 or higher AEs; two had elevated blood creatine phosphokinase which resolved without treatment and was of unclear etiology. No grade 4 or 5 AEs were observed. TRAEs of any grade occurred in six (29%) patients, two (10%) of which were grade 3. One patient experienced grade 3 maculopapular rash, and another patient experienced grade 3 pneumonitis during neoadjuvant therapy (Table 8); this pneumonitis required treatment with steroids and resulted in a delay of surgery by 13 days according to protocol-defined criteria. Upon resolution of the event, successful surgical resection was performed.

TABLE 7
Summary of neoadjuvant TEAEs (any grade in ≥2 patients)

Total (N = 21)

| AE, n (%) | Any grade | Grade ≥3 |
|---|---|---|
| Any | 20 (95.2) | 7 (33.3) |
| Increased aspartate aminotransferase | 4 (19.0) | 0 |
| Increased blood creatine phosphokinase | 3 (14.3) | 2 (9.5) |
| Constipation | 3 (14.3) | 0 |
| Fatigue | 3 (14.3) | 0 |
| Increased alanine aminotransferase | 2 (9.5) | 0 |
| Hypoalbuminemia | 2 (9.5) | 1 (4.8) |
| Abdominal pain | 2 (9.5) | 0 |
| Rash | 2 (9.5) | 0 |
| Cough | 2 (9.5) | 0 |
| Infusion-related reaction | 2 (9.5) | 0 |
| Upper abdominal pain | 2 (9.5) | 0 |
| Hypocalcaemia | 2 (9.5) | 0 |
| Arthralgia | 2 (9.5) | 0 |
| Anaemia | 2 (9.5) | 0 |

TABLE 8
Summary of neoadjuvant TRAEs

Total (N = 21)

| TRAE, n (%) | Any grade | Grade ≥3 |
|---|---|---|
| Any | 6 (28.6) | 2 (9.5) |
| Fatigue | 3 (14.3) | 0 |
| Infusion-related reaction | 2 (9.5) | 0 |
| Rash | 1 (4.8) | 0 |
| Cough | 1 (4.8) | 0 |
| Pneumonitis | 1 (4.8) | 1 (4.8) |
| Pruritus | 1 (4.8) | 0 |
| Rash maculopapular | 1 (4.8) | 1 (4.8) |

Of the 20 patients whose tumors were evaluable for the primary endpoint, four (20%) had STN, including three (15%) who had complete tumor necrosis (100%) at histopathology. Notably, 7 of the 20 patients resected (35%) had tumor necrosis ≥50%, which is a criterion used by other studies to identify patients with significant post-treatment necrosis (Table 9).

TABLE 9
Pathologic tumor necrosis at resection (n = 20)

| Pathologic tumor necrosis | n (%) |
|---|---|
| STN (>70%) | 4 (20) |
| Complete tumor necrosis (100%) | 3 (15) |
| Tumor necrosis (≥50%) | 7 (35) |

On treatment, presurgical MRI was performed on 20 patients at a median of 24 days following initiation of cemiplimab, and one patient underwent presurgical CT. Three patients achieved PR radiographically per RECIST 1.1 for an ORR of 15%, with all other patients maintaining stable disease.

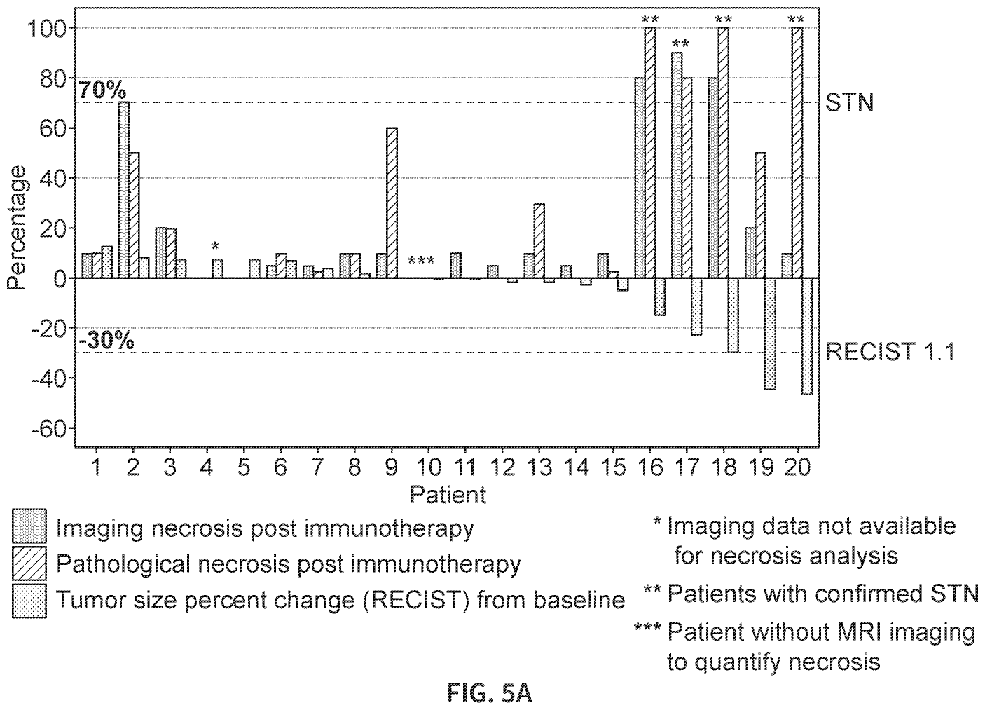

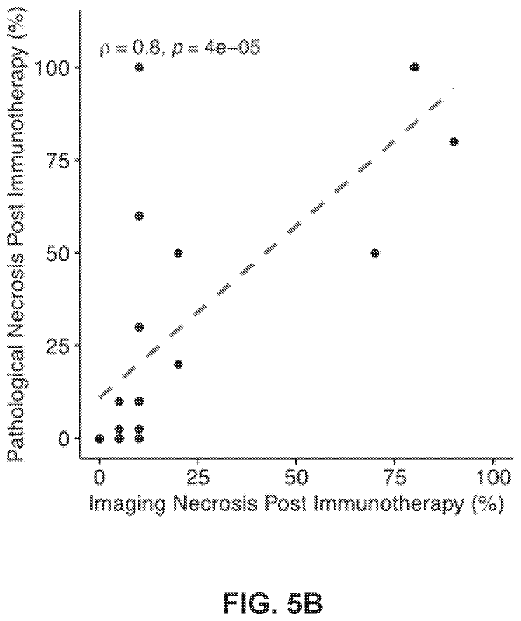

MRI enables estimation of viable tumor based on postcontrast subtracted images, and this technique identified patients with significant necrosis on imaging performed prior to resection, irrespective of radiographic tumor shrinkage. This is illustrated in FIG. 5A, which is a waterfall plot of responses in patients ordered according to increasing response using standard RECIST measurements (dotted line correlates with 30% decrease in tumor size). In parallel, the pathologic assessment of degree of necrosis as assessed by two expert hepatopathologists (based on absolute change in necrosis) and degree of necrosis on MRI done post treatment, prior to surgery (dotted line correlates with 70% necrosis to achieve primary endpoint of STN).

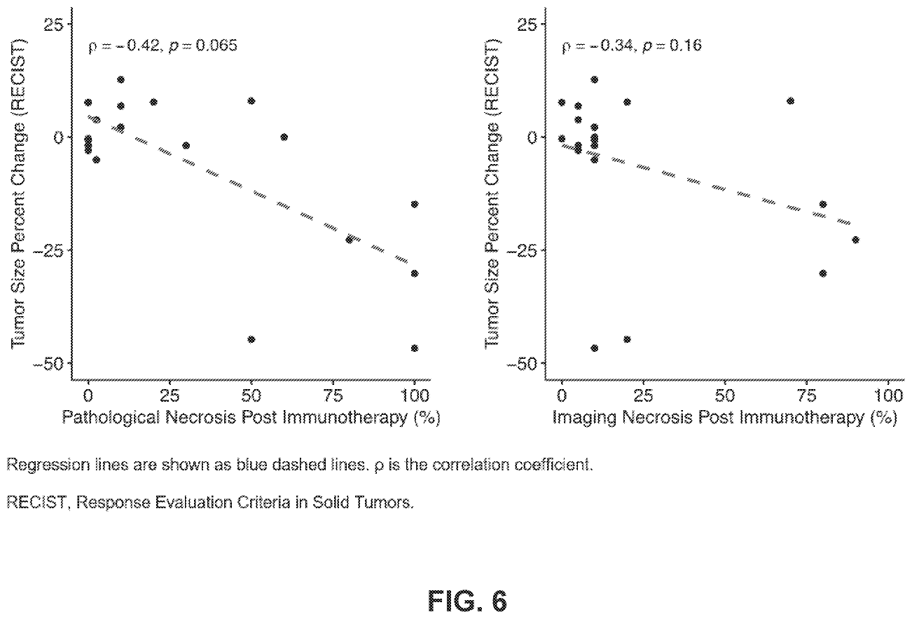

Estimation of necrosis defined by MRI was strongly correlated with pathologic assessment of necrosis at surgery, as illustrated in FIG. 5B (r=0.71-0.72, p<0.0001), which provides a comparative analysis of necrosis measurements by MRI and per pathologic analyses. Regression lines are shown as dashed lines. ρ is the correlation coefficient. In contrast, there was only a moderate correlation between assessment of necrosis (either pathologic or radiographic) and tumor response measured by standard RECIST 1.1, and this did not reach statistical significance (FIG. 6).

Focusing on patients with notable post-treatment necrosis, standard and pathological images from 5 of the 7 patients who achieved ≥50% necrosis highlight examples of radiographic and pathologic necrosis, as summarized in Table 10. Three patients experienced STN on both MRI and pathologic assessment (Patients 16, 17 and 18), however, only one of these patients achieved PR (−30%) by RECIST 1.1 (Patient 18), while the other patients' lesions were considered stable disease per the standard response. Representative pre- and post-treatment imaging and pathologic specimens of 5 patients in whom necrosis was seen upon surgery, highlight heterogeneity of tissue and radiographic findings. Significant shrinkage of the tumor bed was observed in each of these patients. TILs scored by pathologists assessing all tumor regions sampled as 0 (no TILs), 1 (1-2 foci), 2 (≥3 foci) or 3 (diffuse sheets of TILs). Similarly, tertiary lymphoid aggregates scored as 0 (none), 1 (1 seen) 2 (2 seen) 3 (≥3 present). Patient 2, who also had significant necrosis on resection, had even higher baseline necrosis on pretreatment biopsy, and patient 20 did not have tumor cells present in pretreatment biopsy.

TABLE 10
Representative Tissue analysis and mIHC

| | Patient 9 | Patient 16 | Patient 17 | Patient 18 | Patient 19 |
|---|---|---|---|---|---|
| Pre-Treatment | Necrosis in | Necrosis in | Necrosis in | Necrosis in | Necrosis in |
| MRI | tumor bed: 5% | tumor bed: 30% | tumor bed: 50% | tumor bed: 30% | tumor bed: 0% |
| Pre-Treatment | Necrosis: 5% | Necrosis: 30% | Necrosis: 50% | Necrosis: 30% | Necrosis: 0% |
| Biopsy | TILs: 1 | TILs: 3 | TILs: 0 | TILs: 1 | TILs: 2 |
| | TLS: 2 | TLS: 2 | TLS: 0 | TLS: 0 | TLS: 1 |
| Post-Treatment | Radiographic | Radiographic | Radiographic | Radiographic | Radiographic |
| MRI | necrosis: 0% | necrosis: 50% | necrosis: 90% | necrosis: 70% | necrosis: 20% |
| | RECIST 1.1 | RECIST 1.1 | RECIST 1.1 | RECIST 1.1 | RECIST 1.1 |
| | change: −0% | change: −15% | change: −22% | change: −30% | change: −45% |
| Post-Treatment | Significant | Significant | Significant | Significant | Significant |
| Gross | shrinkage of | shrinkage of | shrinkage of | shrinkage of | shrinkage of |
| Specimen | tumor bed | tumor bed | tumor bed | tumor bed | tumor bed |
| Post-Treatment | Necrosis: 60% | Necrosis: 100% | Necrosis: 80% | Necrosis: 100% | Necrosis: 50% |
| Pathology | TILs: 3 | TILs: 3 | TILs: 3 | TILs: 3 | TILs: 3 |
| | TLS: 2 | TLS: 2 | TLS: 2 | TLS: 2 | TLS: 2 |

For exploratory tissue analysis, a comparison was conducted between the seven patients with ≥50% histopathologic necrosis and the remaining 13 patients who had undergone resection and were found to have little to no necrosis in their resected tumors, all 30% or less (Table 11). Six of the seven patients identified using this exploratory cut-off had an increase in necrosis seen on their baseline biopsy, suggestive of a therapeutic effect (Table 11). One of these seven patients, Patient 2, had a highly necrotic tumor at baseline and had no appreciable change in level of necrosis following treatment on MRI or pathologic examination, and the tumor size was slightly increased while on therapy. Of the seven patients with ≥50% necrosis in this trial, three had a history of HBV, two had nonalcoholic steatohepatitis/nonalcoholic fatty liver disease (NASH/NAFLD), one had HCV-related cirrhosis, and one had alcoholic cirrhosis (Table 11).

TABLE 11
Individual patient data on response and underlying etiology of HCC

| | | | | Imaging (%) | | |
|---|---|---|---|---|---|---|
| | | | | | | Tumor size |
| | Pathology (%) | | | | | change per |
| Patient | HCC risk | Biopsy | Resection | Necrosis pre- | Necrosis post | RECIST 1.1 on |
| number | factor | necrosis | necrosis | immunotherapy | immunotherapy | presurgical MRI |
| P1 | HBV | 10 | 10 | 5 | 10 | 12.71 |
| P2 | HBV | 60 | 50 | 70 | 70 | 8 |
| P3 | NASH | 0 | 20 | 20 | 20 | 7.75 |
| P4 | HBV | 0 | 0 | 0 | 0 | 7.69 |
| P5 | HCV | 0 | 0 | n/a | n/a | 7.69 |
| P6 | None | 0 | 10 | 0 | 0 | 6.9 |
| P7 | HCV | 0 | <5 | 0 | 0 | 3.85 |
| P8 | NASH | 0 | 10 | 10 | 10 | 2.18 |
| P9 | HCV | 5 | 60 | 0 | 0 | 0 |
| P10 | NASH | 0 | 0 | 0 | 0 | −0.35 |
| P11 | HCV | 0 | 0 | 10 | 10 | −0.62 |
| P12 | HCV | 0 | 0 | 0 | 5 | −1.83 |
| P13 | HBV | 80 | 30 | 5 | 5 | −1.85 |
| P14 | HBV | n/a | 0 | 5 | 5 | −2.9 |
| P15 | HBV | 10 | <5 | 5 | 5 | −5 |
| P16 | ALD | 30 | 100 | 0 | 50 | −14.85 |
| P17 | NASH | 50 | 80 | 50 | 90 | −22.71 |
| P18 | HBV | 30 | 100 | 20 | 70 | −30.14 |
| P19 | NAFLD | 0 | 50 | 10 | 20 | −44.72 |
| P20 | HBV | n/a | 100 | 5 | 10 | −46.67 |

ALD, alcoholic liver disease; HBV, hepatitis B virus; HCC, hepatocellular carcinoma; HCV, hepatitis C virus; MRI, magnetic resonance imaging; NAFLD, nonalcoholic fatty liver disease; NASH, nonalcoholic steatohepatitis; n/a, not available; RECIST, Response Evaluation Criteria in Solid Tumors.

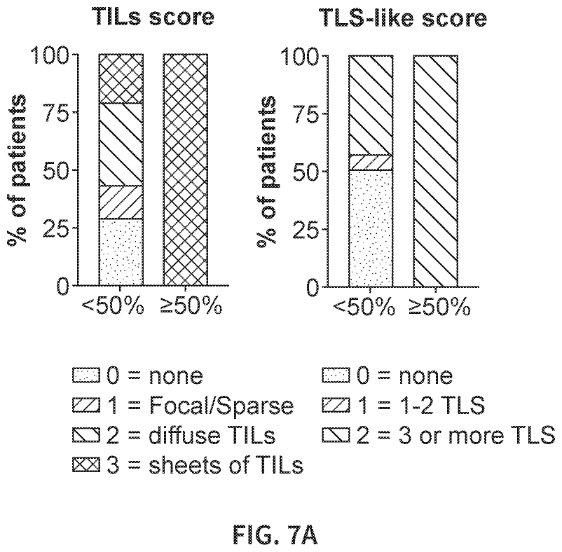

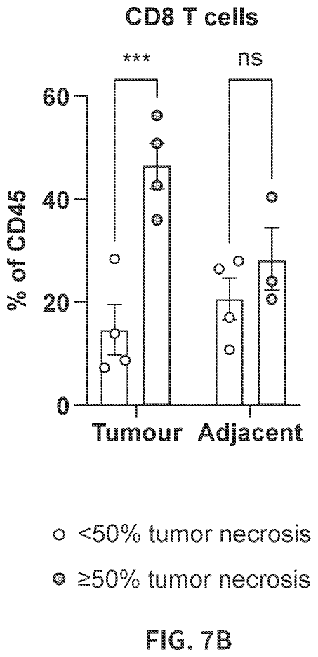

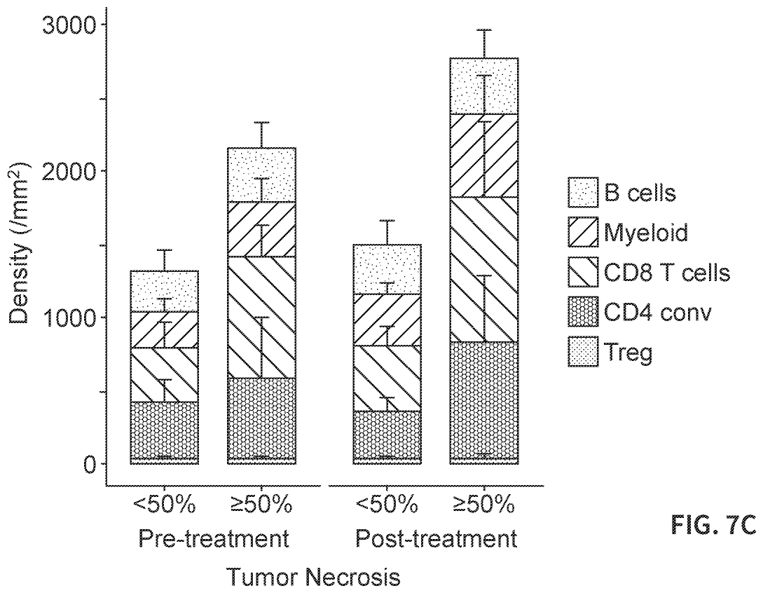

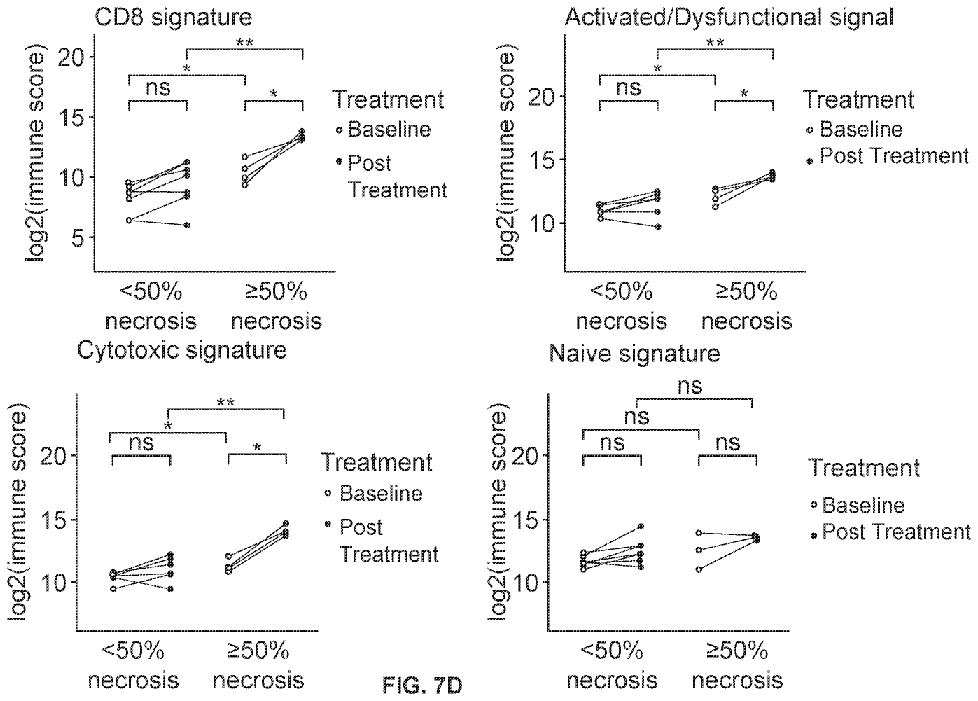

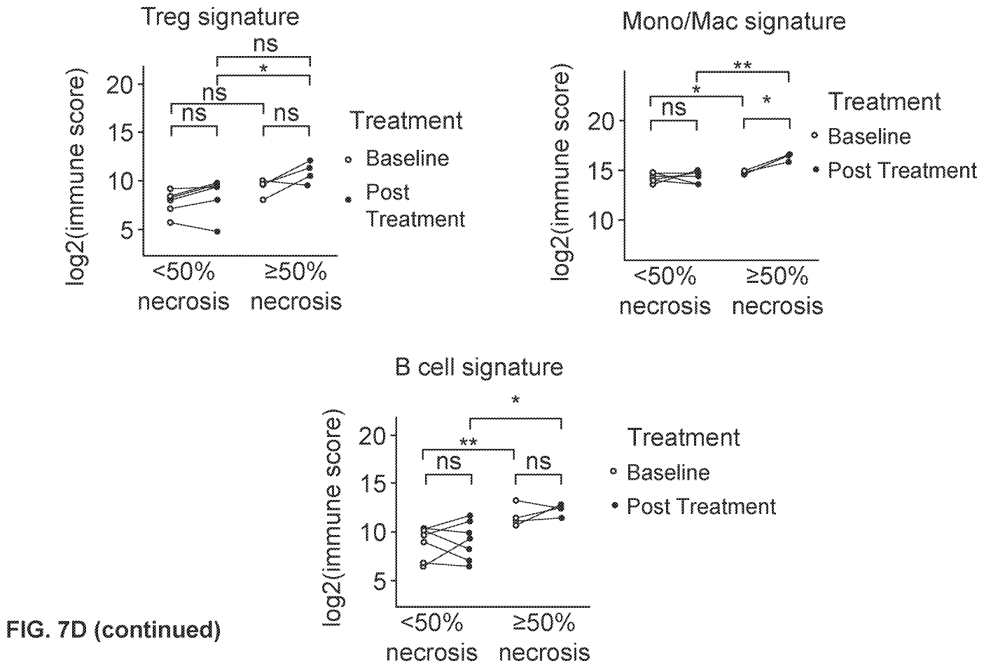

Immunohistochemical analysis of post-treatment lesions showed increased density of immune infiltrates and enhanced TLS-like structures and TILs (FIG. 7A) in patients with ≥50% necrosis compared with patients with little or no necrosis. On post-treatment surgical specimens, all patients who had ≥50% necrosis had the highest TIL infiltration score and high levels of TILs, compared with only 21% of patients who had <50% necrosis; and 29% of patients with <50% necrosis lacked any TIL infiltration. In a subset of patients with adequate tumor for analysis by mass cytometry, four patients with ≥50% necrosis (three patients with 100% necrosis and one patient with 50% necrosis) had significantly higher CD8+ T-cell infiltration in the tumor compared with four patients with little to no necrosis; this finding was unique to the tumor as similar numbers of T cells were seen in the noninvolved adjacent margin that was resected (FIG. 7B).

Further, mlHC of pre- and post-treatment specimens, quantifying immune infiltrate in multiple representative regions of interest, showed enhanced immune infiltrates at baseline, which further increased after therapy in patients who had ≥50% necrosis. These were relatively unchanged in patients who had minimal to no necrosis following treatment, though given interpatient variability this finding did not reach statistical significance (FIG. 7C). An mlHC panel (CD3-CD8-FOXP3-CD68-CD20) was used to perform quantitative image analysis and measure density of each immune subset shown in FIG. 7C.

In BulkSeq analyses of RNA from paired pre- and post-treatment specimens, published signatures for CD8+ T cells, activated/dysfunctional (exhausted) cells, cytotoxic cells, monocyte-derived macrophages (Mono/Mac), and B cells were all enriched at baseline in patients who subsequently were found to have ≥50% necrosis upon resection, and all but the B cell signature increased following therapy in these patients, while there was no change seen in patients with little to no necrosis in the expression levels of any of these signatures (<50%) (FIG. 7D). Additionally, a heat map representation of Bulk RNA sequencing (BulkSeq) of biopsy cores and tumor resection of 11 patients (7 patients with little to no necrosis on resection [all <50% necrosis] and 4 patients with ≥50% necrosis) was observed.

In FIGS. 7A-7D, *p<0.05; **p<0.01; Conv, conventional; DAPI, 4′,6-diamidino-2-phenylindole; FFPE, formalin fixed and paraffin embedded; FOXP, Forkhead box protein; H&E, haematoxylin and eosin; HCC, hepatocellular carcinoma; MICSSS, Multiplexed Immunohistochemical Consecutive Staining on Single Slide; mIHC, multiplex immunohistochemistry; ns, not significant; TIL, tumor-infiltrating lymphocyte; TLS, tertiary lymphoid structure; Treg, regulatory T cell.

Conclusions: This study demonstrates that a short course of neoadjuvant cemiplimab resulted in pathologic responses in patients with resectable HCC. The safety profile of cemiplimab was acceptable. In initial pathologic assessments and based on the sequencing from pre- and post-treatment tissue, there was a positive correlation between molecular signatures of tumour immune activity and pathologic necrosis, as well as a correlation between increase in immune infiltrate response from baseline and greater pathologic necrosis. In addition, while standard imaging response criteria (RECIST 1.1) fails to identify pathologic responses in most patients after a brief course of therapy, contrast-enhanced MRI was demonstrated to be an accurate noninvasive method to assess tumor necrosis in response to therapy and should be used in conjunction with RECIST 1.1 to quantify total change in viable tumor following therapy.

In this trial, which is believed to be the largest trial to date of perioperative PD-1 targeted monotherapy in HCC, cemiplimab demonstrated clinical activity in a patient population with an unmet clinical need. Further, an STN rate of 20%, with a total of 35% of patients having ≥50% tumor necrosis at surgery was observed, along with a 10% rate of perioperative grade 3 TRAEs. In this HCC patient population in whom surgery is a curative intent therapy, cemiplimab neadjuvant therapy provides a substantial advantage over other treatments that require a longer period of induction therapy prior to surgery which may increase the likelihood of perioperative toxicity and may also delay or preclude surgery.

Across the 7 patients who had ≥50% necrosis in this trial, all etiologies were represented; three had a history of HBV, two had NASH/NAFLD, one had HCV-related cirrhosis, and one had alcoholic cirrhosis (Table 11). The pathologic response seen in two patients with NASH/NAFLD is notable given our finding that patients with NASH-related HCC fared significantly worse than patients with HCC from other etiologies (Pfister et al., Nature, 2021; 592(7854):450-56). For one patient (Patient 17) who had a confirmed diagnosis of NASH, there were no documented TILs or TLS-like structures on the pretreatment biopsy, while a robust immune infiltrate was seen on the excised tumor, suggesting that NASH-related HCC may be responsive to immunotherapy, at least in the early-stage setting.

The immune infiltrate in patients whose tumors had ≥50% necrosis was more robust than in patients with little or no necrosis on surgical samples following treatment with cemiplimab. Additionally, the density of immune infiltrate in pretreatment biopsy based on BulkSeq data correlates with this higher necrosis post treatment, complemented by trends observed from mlHC, suggesting that patients with an underlying immune recognition of their tumor are more likely to respond to PD-1 blockade monotherapy.

### REFERENCES

1. Akateh C et al. World J Gastroenterol 2019; 25:3704-3721.
2. Alexandrov et al., “Signatures of mutational processes in human cancer,” Nature, 2013; 500(7463):415-421.
3. Allard et al., “Does pathological response after transarterial chemoembolization for hepatocellular carcinoma in cirrhotic patients with cirrhosis predict outcome after liver resection or transplantation?” J Hepatol, 2015; 63(1):83-92.
4. Anagnostou et al., “Evolution of neoantigen landscape during immune checkpoint blockade in non-small cell lung cancer,” Cancer Discov, 2017; 7(3):264-276.
5. Ang et al., Human papillomavirus and survival of patients with oropharyngeal cancer. N Engl J Med. 2010; 363(1):24-35.
6. Angelo et al., “Multiplexed ion beam imaging of human breast tumors,” Nat Med, 2014; 20(4):436-442.
7. Antonia et al., “Durvalumab after Chemoradiotherapy in Stage Ill Non-Small-Cell Lung Cancer,” N Engl J Med, 2017; 377:1919-1929.
8. Bauml et al., “Pembrolizumab for Platinum- and Cetuximab-Refractory Head and Neck Cancer: Results From a Single-Arm, Phase II Study,” Journal of Clinical Oncology: Official Journal of the American Society of Clinical Oncology, 2017:Jco2016701524.
9. Blanchard et al., “Meta-analysis of chemotherapy in head and neck cancer (MACH-NC): a comprehensive analysis by tumour site,” Radiother Oncol, 2011; 100(1):33-40.
10. Blank et al., Nat Med 2018; 24(11): 1655-61.
11. Borghaei et al., “Nivolumab versus Docetaxel in Advanced Nonsquamous Non-Small-Cell Lung Cancer,” N Engl J Med, 2015; 373(17):1627-1639.
12. Bruix et al., “Adjuvant sorafenib for hepatocellular carcinoma after resection or ablation (STORM): a phase 3, randomised, double-blind, placebo-controlled trial,” The Lancet Oncology, 2015; 16(13):1344-1354.
13. Burova E et al. Mol Cancer Ther. 2017; 16:861-870.
14. Chaft et al., “Neoadjuvant nivolumab in early-stage, resectable non-small cell lung cancers, Journal of Clinical Oncology, 2017; 35(15_suppl):8508-8508.
15. Chalabi et al., Nat Med 2020; 26(4): 566-76.
16. Chan A C et al. Liver Transpl. 2013; 19:411-419.
17. Chan K M et al., Ann Surg Oncol 2011; 18(9): 2638-46.
18. Chaturvedi et al., “Incidence trends for human papillomavirus-related and -unrelated oral squamous cell carcinomas in the United States,” Journal of Clinical Oncology: Official Journal of the American Society of Clinical Oncology, 2008; 26(4):612-619.
19. Chowell et al., “Patient HLA class I genotype influences cancer response to checkpoint blockade immunotherapy,” Science, 2018; 359(6375):582-587.
20. Danilova et al., “Association of PD-1/PD-L axis expression with cytolytic activity, mutational load, and prognosis in melanoma and other solid tumors,” Proc Natl Acad Sci USA, 2016; 113(48):E7769-E7777.
21. de Martel et al., “Global burden of cancers attributable to infections in 2008: a review and synthetic analysis,” The Lancet Oncology, 2012; 13(6):607-615.
22. Eisenhauer et al., “New response evaluation criteria in solid tumours: revised RECIST guideline (version 1.1),” Eur J Cancer, 2009; 45(2):228-247.
23. EI-Khoueiry et al., “Nivolumab in patients with advanced hepatocellular carcinoma (CheckMate 040): an open-label, non-comparative, phase 1/2 dose escalation and expansion trial,” Lancet, 2017; 389(10088):2492-2502.
24. European Association for the Study of the Liver. J Hepatol. 2018; 69:182-236.
25. Fehrenbacher et al., “Atezolizumab versus docetaxel for patients with previously treated non-small-cell lung cancer (POPLAR): a multicentre, open-label, phase 2 randomised controlled trial,” Lancet, 2016; 387(10030):1837-1846.
26. Ferlay et al., “Cancer incidence and mortality worldwide: Sources, methods and major patterns in GLOBOCAN 2012,” International Journal of Cancer, 2015; 136(5): E359-E386.
27. Forde et al., “Neoadjuvant PD-1 Blockade in Resectable Lung Cancer,” N Engl J Med, 2018; 378:1976-86.
28. Franssen et al., “Differences in surgical outcomes between hepatitis B- and hepatitis C-related hepatocellular carcinoma: a retrospective analysis of a single North American center,” Ann Surg, 2014; 260(4):650-656.
29. Garon et al., “Pembrolizumab for the treatment of non-small-cell lung cancer,” N Engl J Med, 2015; 372(21):2018-2028.
30. Gordic et al., J Hepatol 2017; 67(6): 1213-21.
31. Group et al., “Adjuvant chemotherapy, with or without postoperative radiotherapy, in operable non-small-cell lung cancer: two meta-analyses of individual patient data,” Lancet, 2010; 375(9722):1267-1277.
32. Group NM-aC, “Preoperative chemotherapy for non-small-cell lung cancer: a systematic review and meta-analysis of individual participant data,” Lancet, 2014; 383(9928):1561-1571.
33. Hectors et al., “Quantification of hepatocellular carcinoma heterogeneity with multiparametric magnetic resonance imaging,” Sci Rep, 2017; 7(1):2452.
34. Hellmann et al., “Pathological response after neoadjuvant chemotherapy in resectable non-small-cell lung cancers: proposal for the use of major pathological response as a surrogate endpoint,” The Lancet Oncology, 2014; 15(1): e42-50.
35. Ho et al., “The mutational landscape of adenoid cystic carcinoma,” Nat Genet, 2013; 45(7):791-798.
36. Hoffman et al., “Orbital Exenteration to Manage Infiltrative Sinonasal, Orbital Adnexal, and Cutaneous Malignancies Provides Acceptable Survival Outcomes: An Institutional Review, Literature Review, and Meta-Analysis,” J Oral Maxillofac Surg, 2016; 74(3):631-43.
37. Howlader et al., SEER Cancer Statistics Review, 1975-2014, based on November 2016 SEER data submission, posted to the SEER website, April 2017. Bethesda, MD: National Cancer Institute.
38. Huang et al., Nat Med 2019; 25(3):454-61.
39. Huang et al., Nature 2017; 545(7652): 60-65.
40. Jung et al., Cancer Res Treat, 2017; 49(1): 246-54.
41. Kaseb et al., J Clin Oncol 2020; 38(4_suppl): 486.
42. Langer et al., “Carboplatin and pemetrexed with or without pembrolizumab for advanced, non-squamous non-small-cell lung cancer: a randomised, phase 2 cohort of the open-label KEYNOTE-021 study,” The Lancet Oncology, 2016; 17(11):1497-1508.
43. Lavin et al., “Innate Immune Landscape in Early Lung Adenocarcinoma by Paired Single-Cell Analyses,” Cell, 2017; 169(4):750-765 e717.
44. Lei et al., “Analyses of PD-L1 and inflammatory gene expression association with efficacy of nivolumab±ipilimumab in gastric cancer/gastroesophageal junction cancer,” Clinical Cancer Research 2021.
45. Levrero et al., “Mechanisms of HBV-induced hepatocellular carcinoma,” J Hepatol, 2016; 64(1 Suppl): S84-S101.
46. Li et al., MedComm 2021; 2(1): 60-68.
47. Libtayo package insert, https://www.libtayohcp.com/studydesign (2020).
48. Liu et al., “Improved Efficacy of Neoadjuvant Compared to Adjuvant Immunotherapy to Eradicate Metastatic Disease,” Cancer Discov, 2016; 6(12):1382-1399.
49. Łuksza et al., “A neoantigen fitness model predicts tumour response to checkpoint blockade immunotherapy,” Nature, 2017; 551(7681):517-520.
50. Martini et al., “Incidence of local recurrence and second primary tumors in resected stage I lung cancer,” J Thorac Cardiovasc Surg, 1995; 109(1):120-129.
51. Murakami et al., Exp Ther Med 2010; 1(3): 485-91.
52. Naidoo et al., “Toxicities of the anti-PD-1 and anti-PD-L1 immune checkpoint antibodies,” Ann Oncol, 2015; 26:2375-2391.
53. National Comprehensive Cancer Network. Clinical practice guidelines in oncology: hepatobilliary cancers version 3.2021 (Jun. 15, 2021)
54. Nishino et al., “Developing a common language for tumor response to immunotherapy: immune-related response criteria using unidimensional measurements,” Clin Canc Res, 2013; 19:3936-3943.
55. Pataer et al., “Histopathologic response criteria predict survival of patients with resected lung cancer after neoadjuvant chemotherapy,” J Thorac Oncol, 2012; 7(5):825-832.
56. Paz-Ares et al., “Phase 3 study of carboplatin-paclitaxel/nab-paclitaxel (Chemo) with or without pembrolizumab (Pembro) for patients (Pts) with metastatic squamous (Sq) non-small cell lung cancer (NSCLC),” American Society of Clinical Oncology; 2018.
57. Pignon et al., “Lung adjuvant cisplatin evaluation: a pooled analysis by the LACE Collaborative Group,” Journal of Clinical Oncology: Official Journal of the American Society of Clinical Oncology, 2008; 26(21):3552-3559.
58. Pinato et al., Hepatology 2020; doi: 10.1002/hep.31697 (online ahead of print).
59. Pfister et al., Nature 2021; 592(7854): 450-56.
60. Poon R et al. Ann Surg. 2000; 232:10-24.
61. Provencio-Pulla et al., “Neoadjuvant chemo/immunotherapy for the treatment of stages IIIA resectable non-small cell lung cancer (NSCLC): A phase II multicenter exploratory study-NADIM study-SLCG,” J Clin Onc, 2018; 36, 15 Suppl 8521.
62. Regeneron Pharmaceuticals, Inc. LIBTAYO® [cemiplimab-rwlc]injection full US prescribing information (2021).
63. Remark et al., “In-depth tissue profiling using multiplexed immunohistochemical consecutive staining on single slide,” Science Immunology, 2016; 1(1):6925.
64. Riaz et al., “Tumor and Microenvironment Evolution during Immunotherapy with Nivolumab,” Cell, 2017; 171(4):934-949 e915.
65. Rizvi et al., “Cancer immunology. Mutational landscape determines sensitivity to PD-1 blockade in non-small cell lung cancer,” Science, 2015; 348(6230):124-128.
66. Rusch et al., “Neoadjuvant atezolizumab in resectable non-small cell lung cancer (NSCLC): Initial results from a multicenter study (LCMC3),” Journal of Clinical Oncology, 2018; 36(15 Suppl):8541.
67. Salmon et al., “Expansion and Activation of CD103(+) Dendritic Cell Progenitors at the Tumor Site Enhances Tumor Responses to Therapeutic PD-L1 and BRAF Inhibition,” Immunity, 2016; 44(4):924-938.
68. Sampson et al., “Second symposium on the definition and management of anaphylaxis: summary report—second National Institute of Allergy and Infectious Disease/Food Allergy and Anaphylaxis Network symposium,” Ann Emerg Med, 2006 April; 47(4):373-80.
69. Schmid et al., N Engl J Med 2020; 382(9): 810-21.
70. Shindoh et al., HPB (Oxford) 2013; 15(1): 31-39.
71. Shu et al., “Neoadjuvant atezolizumab+ chemotherapy in resectable non-small cell lung cancer (NSCLC),” J Clin Oncol, 2018; 36:8532.
72. Siegel et al., “CA: A Cancer Journal for Clinicians,” Cancer statistics, 2013, 2013; 63(1):11-30.
73. Snyder et al., “Genetic basis for clinical response to CTLA-4 blockade in melanoma,” N Engl J Med, 2014; 371(23):2189-2199.
74. Strauss et al., “Adjuvant paclitaxel plus carboplatin compared with observation in stage IB non-small-cell lung cancer: CALGB 9633 with the Cancer and Leukemia Group B, Radiation Therapy Oncology Group, and North Central Cancer Treatment Group Study Groups,” Journal of Clinical Oncology: Official Journal of the American Society of Clinical Oncology, 2008; 26(31):5043-5051.
75. Sung et al. C A Cancer J Clin. 2021; 71(3):209-49.
76. Szabo et al., Nat Commun 2019; 10(1): 1-16.
77. Tabrizian et al., “Recurrence of hepatocellular cancer after resection: patterns, treatments, and prognosis,” Ann Surg, 2015; 261(5):947-955.
78. Temple R., “Hy's law: predicting serious hepatotoxicity,” Pharmacoepidemiol Drug Saf, 2006 April; 15(4):241-3.
79. Tirumani et al., “Radiographic Profiling of Immune-Related Adverse Events in Advanced Melanoma Patients Treated with Ipilimumab,” Cancer Immunol Res, 2015; 3(10):1185-92.
80. Topalian et al., Science 2020; 367(6477): eaax0182.
81. Topalian et al., JAMA Oncol 2019; 5: 1411-20.
82. Torre et al., “CA: A Cancer Journal for Clinicians,” Global cancer statistics, 2012, 2015; 65(2):87-108.
83. Uppaluri et al., “Neoadjuvant pembrolizumab in surgically resectable, locally advanced HPV negative head and neck squamous cell carcinoma (HNSCC),” Journal of Clinical Oncology, 2017; 35(15_suppl):6012-6012.
84. Van der Leun et al., Nat Rev Cancer 2020; 20(4): 218-32.
85. Wakelee et al., “E1505: Adjuvant chemotherapy+/−bevacizumab for early stage NSCLC-Outcomes based on chemotherapy subsets,” Journal of Clinical Oncology, 2016; 34(15_suppl):8507-8507.
86. Weber et al., “Adjuvant Nivolumab versus Ipilimumab in Resected Stage Ill or IV Melanoma,” N Engl J Med, 2017; 377(19):1824-1835.
87. Weber et al., “Toxicities of immunotherapy for the practitioner,” J Clin Oncol, 2015; 33:2092-9.
88. Wise-Draper et al., “Phase II multi-site investigation of neoadjuvant pembrolizumab and adjuvant concurrent radiation and pembrolizumab with or without cisplatin in resected head and neck squamous cell carcinoma,” J Clin Oncol, 2018; 36(Suppl. 15):6017.
89. Yarchoan et al., “Characterization of the Immune Microenvironment in Hepatocellular Carcinoma,” Clin Cancer Res, 2017; 23(23):7333-7339.
90. Zhang et al., Lab Investig 2017; 97(7): 873-85.
91. Zimmer et al., “Neurological, respiratory, musculoskeletal, cardiac and ocular side-effects of anti-PD-1 therapy,” Eur J Cancer, 2016; 60 210e225.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method of treating or inhibiting the growth of a tumor, comprising:
(a) selecting a patient with liver cancer;
(b) administering to the patient a therapeutically effective amount of a neoadjuvant programmed death-1 (PD-1) inhibitor, wherein the neoadjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2; and
(c) after step (b), surgically resecting the liver cancer tumor.

2. The method according to claim 1, wherein the liver cancer is resectable.

3. The method according to claim 1, wherein the liver cancer is selected from hepatocellular carcinoma (HCC), fibrolamellar carcinoma, cholangiocarcinoma, angiosarcoma, and hepatoblastoma.

4. The method according to claim 1, wherein the liver cancer is HCC.

5. The method according to claim 1, wherein the liver cancer is recurrent.

6. The method according to claim 1, wherein the liver cancer is metastatic.

7. The method according to claim 1, wherein the patient has liver cancer for which the intent of surgery would be curative.

8. The method according to claim 1, wherein the patient has a chronic viral infection that has been treated and controlled with an anti-viral therapy and wherein the chronic viral infection comprises HIV, HBV, HCV, or a combination thereof.

9. The method according to claim 1, wherein the patient has squamous or non-squamous liver cancer.

10. The method according to claim 1, wherein the patient has PD-L1 expression in ≥1% of liver cancer cells.

11. The method according to claim 1, wherein surgical resection is performed more than 28 days after step (b).

12. The method according to claim 1, wherein the administered neoadjuvant anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

13. The method according to claim 1, wherein the administered neoadjuvant anti-PD-1 antibody comprises a HCVR comprising an amino acid sequence of SEQ ID NO: 1.

14. The method according to claim 1, wherein the administered neoadjuvant anti-PD-1 antibody comprises a LCVR comprising an amino acid sequence of SEQ ID NO: 2.

15. The method according to claim 1, wherein the administered neoadjuvant anti-PD-1 antibody comprises a HCVR/LCVR amino acid sequence pair of SEQ ID NOs: 1/2.

16. The method according to claim 1, wherein the administered neoadjuvant anti-PD-1 antibody comprises a heavy chain and a light chain, wherein the heavy chain has an amino acid sequence of SEQ ID NO: 9.

17. The method according to claim 1, wherein the administered neoadjuvant anti-PD-1 antibody comprises a heavy chain and a light chain, wherein the light chain has an amino acid sequence of SEQ ID NO: 10.

18. The method according to claim 1, wherein the administered neoadjuvant anti-PD-1 antibody comprises a heavy chain and a light chain, wherein the heavy chain has an amino acid sequence of SEQ ID NO: 9 and the light chain has an amino acid sequence of SEQ ID NO: 10.

19. The method according to claim 1, wherein the administered neoadjuvant anti-PD-1 antibody is cemiplimab.

20. The method according to claim 1, wherein the administered neoadjuvant PD-1 inhibitor is an anti-PD-1 antibody comprising a HCVR with 90%, 95%, 97%, or 98% sequence identity to SEQ ID NO: 1.

21. The method according to claim 1, wherein the administered neoadjuvant PD-1 inhibitor is an anti-PD-1 antibody comprising a LCVR with 90%, 95%, 97%, or 98% sequence identity to SEQ ID NO: 2.

22. The method according to claim 1, wherein the administered neoadjuvant PD-1 inhibitor is an anti-PD-1 antibody comprising a HCVR with 90%, 95%, 97%, or 98% sequence identity to SEQ ID NO: 1, and a LCVR with 90%, 95%, 97%, or 98% sequence identity to SEQ ID NO: 2.

23. The method according to claim 1, further comprising:
(d) after step (c), administering to the patient a therapeutically effective amount of an adjuvant programmed death-1 (PD-1) inhibitor, wherein the adjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.

24. The method according to claim 23, wherein the administered adjuvant anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

25. The method according to claim 23, wherein the administered adjuvant anti-PD-1 antibody comprises a HCVR comprising an amino acid sequence of SEQ ID NO: 1.

26. The method according to claim 23, wherein the administered adjuvant anti-PD-1 antibody comprises a LCVR comprising an amino acid sequence of SEQ ID NO: 2.

27. The method according to claim 23, wherein the administered adjuvant anti-PD-1 antibody comprises a HCVR/LCVR amino acid sequence pair of SEQ ID NOs: 1/2.

28. The method according to claim 23, wherein the administered adjuvant anti-PD-1 antibody comprises a heavy chain and a light chain, wherein the heavy chain has an amino acid sequence of SEQ ID NO: 9.

29. The method according to claim 23, wherein the administered adjuvant anti-PD-1 antibody comprises a heavy chain and a light chain, wherein the light chain has an amino acid sequence of SEQ ID NO: 10.

30. The method according to claim 23, wherein the administered adjuvant anti-PD-1 antibody comprises a heavy chain and a light chain, wherein the heavy chain has an amino acid sequence of SEQ ID NO: 9 and the light chain has an amino acid sequence of SEQ ID NO: 10.

31. The method according to claim 23, wherein the administered adjuvant PD-1 inhibitor is an anti-PD-1 antibody comprising a HCVR with 90%, 95%, 97%, or 98% sequence identity to SEQ ID NO: 1.

32. The method according to claim 23, wherein the administered adjuvant PD-1 inhibitor is an anti-PD-1 antibody comprising a LCVR with 90%, 95%, 97%, or 98% sequence identity to SEQ ID NO: 2.

33. The method according to claim 23, wherein the administered adjuvant PD-1 inhibitor is an anti-PD-1 antibody comprising a HCVR with 90%, 95%, 97%, or 98% sequence identity to SEQ ID NO: 1, and a LCVR with 90%, 95%, 97%, or 98% sequence identity to SEQ ID NO: 2.

34. The method according to claim 1, wherein the method leads to necrosis of the resected tumor, promotes tumor regression, reduces tumor cell load, reduces tumor burden, and/or prevents tumor recurrence in the patient.

35. The method according to claim 1, wherein the method leads to more than 50% necrosis of the resected tumor.

36. The method according to claim 1, wherein the method leads to more than 70% necrosis of the resected tumor.

37. The method according to claim 1, further comprising administering to the patient an additional therapeutic agent or therapy selected from one or more of: an anti-viral therapy, photodynamic therapy, a programmed death ligand 1 (PD-L1) inhibitor, a lymphocyte activation gene 3 (LAG3) inhibitor, a cytotoxic T-lymphocyte-associated protein 4 (CTLA-4) inhibitor, a glucocorticoid-induced tumor necrosis factor receptor (GITR) agonist, a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand, a CD20 inhibitor, an indoleamine-2,3-dioxygenase (IDO) inhibitor, a CD28 activator, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an agonist to a co-stimulatory receptor, an antibody to a tumor-specific antigen, a vaccine, an adjuvant to increase antigen presentation, an oncolytic virus, a cytotoxin, a chemotherapeutic agent, platinum-based chemotherapy, a tyrosine kinase inhibitor, an IL-6R inhibitor, an IL-4R inhibitor, an IL-10 inhibitor, a cytokine, an antibody drug conjugate (ADC), chimeric antigen receptor T cells, an anti-inflammatory drug, and a dietary supplement.

38. The method of claim 1, wherein the neoadjuvant PD-1 inhibitor is administered as one or more doses, wherein each dose is administered every two weeks, three weeks, four weeks, five weeks or six weeks.

39. The method of claim 1, wherein the neoadjuvant PD-1 inhibitor is administered as two or more doses, wherein each dose is administered every three weeks.

40. The method of claim 1, wherein the neoadjuvant PD-1 inhibitor is administered at a dose of 5 mg to 1000 mg.

41. The method of claim 1, wherein the neoadjuvant PD-1 inhibitor is administered at a dose of 200 mg, 250 mg, 350 mg, 400 mg, 500 mg, 600 mg, 750 mg, 800 mg, or 1000 mg.

42. The method of claim 1, wherein the neoadjuvant PD-1 inhibitor is administered at a dose of 1 mg/kg to 20 mg/kg of the patient's body weight.

43. The method of claim 1, wherein the neoadjuvant PD-1 inhibitor is administered at a dose of 1 mg/kg, 3 mg/kg or 10 mg/kg of the patient's body weight.

44. The method according to claim 1, wherein the neoadjuvant PD-1 inhibitor is administered intravenously, or subcutaneously.

45. The method of claim 23, wherein the adjuvant PD-1 inhibitor is administered as one or more doses, wherein each dose is administered every two weeks, three weeks, four weeks, five weeks or six weeks.

46. The method of claim 23, wherein each dose of the adjuvant PD-1 inhibitor is administered every three weeks.

47. The method of claim 23, wherein the adjuvant PD-1 inhibitor is administered at a dose of 5 mg to 1000 mg.

48. The method of claim 23, wherein the adjuvant PD-1 inhibitor is administered at a dose of 200 mg, 250 mg, 350 mg, 400 mg, 500 mg, 600 mg, 750 mg, 800 mg, or 1000 mg.

49. The method of claim 23, wherein the adjuvant PD-1 inhibitor is administered at a dose of 1 mg/kg to 20 mg/kg of the patient's body weight.

50. The method of claim 23, wherein the adjuvant PD-1 inhibitor is administered at a dose of 1 mg/kg, 3 mg/kg or 10 mg/kg of the patient's body weight.

51. The method of claim 23, wherein the adjuvant PD-1 inhibitor is administered intravenously, or subcutaneously.

52. (canceled)

53. A kit comprising a programmed death 1 (PD-1) inhibitor in combination with written instructions for use of a therapeutically effective amount of the PD-1 inhibitor for treating or inhibiting the growth of a tumor in a patient with liver cancer, wherein the PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.

54. A method of treating or inhibiting the growth of a tumor, comprising:
(a) selecting a patient with lung cancer;
(b) administering to the patient a therapeutically effective amount of a neoadjuvant programmed death-1 (PD-1) inhibitor, wherein the neoadjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2; and
(c) after step (b), surgically resecting the lung cancer tumor.

55. The method according to claim 54, wherein the lung cancer is non-small cell lung cancer.

56. The method according to claim 54, wherein the administered neoadjuvant anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

57. The method according to claim 54, wherein the administered neoadjuvant anti-PD-1 antibody comprises a HCVR/LCVR amino acid sequence pair of SEQ ID NOs: 1/2.

58. The method according to claim 54, further comprising:
(d) after step (c), administering to the patient a therapeutically effective amount of an adjuvant programmed death-1 (PD-1) inhibitor, wherein the adjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.

59. A method of treating or inhibiting the growth of a tumor, comprising:
(a) selecting a patient with head and neck cancer;
(b) administering to the patient a therapeutically effective amount of a neoadjuvant programmed death-1 (PD-1) inhibitor, wherein the neoadjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof, and
(c) after step (b), surgically resecting the head and neck cancer tumor.

60. The method according to claim 59, wherein the head and neck cancer is head and neck squamous cell carcinoma.

61. The method according to claim 59, wherein the administered neoadjuvant anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

62. The method according to claim 59, wherein the administered neoadjuvant anti-PD-1 antibody comprises a HCVR/LCVR amino acid sequence pair of SEQ ID NOs: 1/2.

63. The method according to claim 59, further comprising:
(d) after step (c), administering to the patient a therapeutically effective amount of an adjuvant programmed death-1 (PD-1) inhibitor, wherein the adjuvant PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.


SEQUENCE LISTING (10 sequences)

SEQ ID NO: 1 - protein, 117 aa, Artificial Sequence [sequence omitted]

SEQ ID NO: 2 - protein, 107 aa, Artificial Sequence [sequence omitted]

SEQ ID NO: 3 - protein, 8 aa, Artificial Sequence
  HCDR1
GFTFSNFG

SEQ ID NO: 4 - protein, 8 aa, Artificial Sequence
  HCDR2
ISGGGRDT

SEQ ID NO: 5 - protein, 10 aa, Artificial Sequence
  HCDR3
VKWGNIYFDY

SEQ ID NO: 6 - protein, 6 aa, Artificial Sequence
  LCDR1
LSINTF

SEQ ID NO: 7 - protein, 3 aa, Artificial Sequence
  LCDR2
AAS

SEQ ID NO: 8 - protein, 9 aa, Artificial Sequence
  LCDR3
QQSSNTPFT

SEQ ID NO: 9 - protein, 444 aa, Artificial Sequence
  HC
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  LC
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC